(19) United States
(12) Patent Application Publication
(10) Pub. No.: US 2017/0326166 A1
(43) Pub. Date: Nov. 16, 2017

(54) COMPOSITIONS AND METHODS FOR TREATING PITUITARY TUMORS

(71) Applicant: University of Virginia Patent Foundation, Charlottesville, VA (US)

(72) Inventor: Edward H. Oldfield, Charlottesville, VA (US)

(73) Assignee: University of Virginia Patent Foundation, Charlottesville, VA (US)

(21) Appl. No.: 15/519,702

(22) PCT Filed: Oct. 19, 2015
(86) PCT No.: PCT/US15/56250

§ 371 Date: Apr. 17, 2017

Related U.S. Application Data
(60) Provisional application No. 62/065,169, filed on Oct. 17, 2014.

(51) Int. Cl. A61K 31/7004; A61K 38/25; A61K 38/09; A61K 9/00; A61K 38/06; A61K 31/155; A61K 38/28; A61K 38/22

(52) U.S. Cl. CPC A61K 31/7004; A61K 38/28; A61K 38/25; A61K 38/2228; A61K 9/0019; A61K 38/066; A61K 31/155; A61K 38/09

(57) ABSTRACT

The present application discloses that pituitary tumor cells are sensitive to low concentrations of glucose and that methods of treating such tumors include methods to induce infarction that are designed to inhibit glucose uptake, reduce intracellular glucose levels, inhibit glucose utilization, or to reduce available glucose to the tumor or the tumor cells.

### CROSS REFERENCE TO RELATED APPLICATIONS

This application is entitled to priority pursuant to 35 U.S.C. §119(e) to U.S. provisional patent application No. 62/065,169 filed on Oct. 17, 2014. The entire disclosure of the afore-mentioned patent application is incorporated herein by reference.

### BACKGROUND

Pituitary tumors spontaneously infarct at a relatively high rate, higher than any other CNS tumor. This occurs with or without hemorrhage. The typical clinical entity was described relatively late, in 1950, by Brougham et al.¹² Since then pituitary apoplexy has been the subject of many reports describing the clinical presentation, patient management, imaging features, and outcome, as well as reports of acute circumstances predisposing to its occurrence.^{9,10,14,15,17,22-25,29,32,36,37,39,45}

The prior focus on mechanisms underlying pituitary apoplexy has been on these acute events. Less attention has been given to the endogenous features of pituitary tumors that make them susceptible to spontaneous infarction, despite that most pituitary apoplexy occurs in the absence of a recognized precipitating event.

There is a long felt need in the art for compositions and methods useful for treating pituitary tumors. The present invention satisfies this need.

### SUMMARY OF THE INVENTION

Without wishing to be bound by any particular theory, it is hypothesized herein that infarction of pituitary adenomas is the product of a combination of intrinsic features of these tumors and that it is the tenuous imbalance between their high rate of demand for nutrients combined with their limited intrinsic blood supply that makes them vulnerable to infarction, with or without precipitating events, and suggest that this circumstance permits new approaches to treatment based on this peculiar vulnerability.

Pituitary adenomas occasionally undergo infarction, apoplexy, which often destroys much of the tumor. It is well known that apoplexy can be precipitated by several acute factors, including cardiac surgery, other types of surgery, trauma, insulin infusion, and stimulation with administration of hypothalamic releasing factors.

The present application examines intrinsic features of pituitary adenomas that render them vulnerable to apoplexy, features such as high metabolic demand, paucity of angiogenesis, and sparse vascularity, qualities that have previously not been linked with apoplexy, and argue that it is these features of adenomas underlie their susceptibility to spontaneous infarction. To this end, the sensitivity of freshly cultured pituitary adenomas to hypoglycemia was assessed.

Adenomas have high metabolic demand, limited angiogenesis, and reduced vessel density compared to the normal gland. It is disclosed herein that pituitary adenoma cells do not survive in the presence of reduced concentrations of glucose or in the absence of glucose. Therefore, this discovery allows a tumor to be targeted by decreasing glucose levels to an extent that induces infarction of the tumor, but not so much that normal cells are impacted to the extent that the tumor cells are. That is, there exists a differential sensitivity between normal pituitary cells, other normal cells, and the pituitary adenoma cells.

It is proposed herein that the frequent ischemic infarction of pituitary adenomas is the product of intrinsic features of these tumors. These endogenous qualities create a tenuous balance between high metabolic demand and marginal tissue perfusion. Thus, the tumor is vulnerable to spontaneous infarction or to acute ischemia by any event that acutely alters the balance between tumor perfusion and tumor metabolism, events such as acute systemic hypotension, abruptly decreased supply of nutrients, such as hypoglycemia with insulin administration, or increasing the tumor's metabolic demand with administration of hypothalamic releasing factors. The present application discloses compositions and methods that take advantage of these intrinsic features of pituitary adenomas by using aspects of this vulnerability for development of new approaches for treatment.

The present application discloses compositions and methods useful for inducing infarction of pituitary adenomas. The methods vary, and include, for example, administering to a subject an effective amount of an agent that: 1) inhibits glucose uptake or glucose in pituitary adenoma cells; 2) an agent that induces or controls hypoglycemia; 3) an agent that controls systemic hypotension; 4) and a hypothalamic releasing factor. The present invention further includes the use of combinations of these methods.

The present invention provides compositions and methods for selectively treating pituitary tumors, wherein the method induces infarction of pituitary adenoma cells selectively compared to normal cells. The present application discloses that pituitary adenoma tumor cells are sensitive to low concentrations of glucose and that methods of treating such tumors include methods to inhibit glucose uptake, reduce intracellular glucose levels, inhibit glucose utilization, or to reduce available glucose to the tumor and the tumor cells.

In one embodiment, the tumor is identified as being sensitive to low glucose as described herein and based on the identification a treatment regimen is developed for the subject.

In one embodiment, the treatment includes depriving a tumor of glucose. In one aspect, the treatment blocks glucose uptake into the cells. In one aspect, the pituitary tumor is an adenoma. The invention encompasses all methods and reagents for limiting glucose to tumor cells, including reducing blood flow, targeting their glucose transporters, etc.

In one embodiment, an inhibitor of glucose uptake or metabolism is administered to induce infarction of a pituitary adenoma in a subject in need thereof. In one aspect, a deoxyglucose is administered. In one aspect, deoxyglucose is 2-deoxyglucose.

2-deoxyglucose compounds are defined herein as 2-deoxy-D-glucose, and homologs, analogs, and/or derivatives of 2-deoxy-D-glucose. While the levo form is not prevalent, and 2-deoxy-D-glucose is preferred, the term “2-deoxyglucose” is intended to cover inter alia either 2-deoxy-D-glucose and 2-deoxy-L-glucose, or a mixture thereof.

Examples of 2-deoxyglucose compounds useful in the invention are: 2-deoxy-D-glucose, 2-deoxy-L-glucose; 2-bromo-D-glucose, 2-fluoro-D-glucose, 2-iodo-D-glucose, 6-fluoro-D-glucose, 6-thio-D-glucose, 7-glucosyl fluoride, 3-fluoro-D-glucose, 4-fluoro-D-glucose, 1-O-propyl ester of 2-deoxy-D-glucose, 1-O-tridecyl ester of 2-deoxy-D-glucose, 1-O-pentadecyl ester of 2-deoxy-D-glucose, 3-O-propyl ester of 2-deoxy-D-glucose, 3-O-tridecyl ester of 2-deoxy-D-glucose, 3-O-pentadecyl ester of 2-deoxy-D-glucose, 4-O-propyl ester of 2-deoxy-D-glucose, 4-O-tridecyl ester of 2-deoxy-D-glucose, 4-O-pentadecyl ester of 2-deoxy-D-glucose, 6-O-propyl ester of 2-deoxy-D-glucose, 6-O-tridecyl ester of 2-deoxy-D-glucose, 6-O-pentadecyl ester of 2-deoxy-D-glucose, and 5-thio-D-glucose, and mixtures thereof.

In one aspect, 2DG is administered to a subject in need at a dose of about 0.1 mg/kg body weight to about 1.0 g/kg body weight and can be administered for 45 days or less, 30 days or less, 15 days or less, etc. In one aspect, 2DG at about 1.0 mg/kg body weight to about 500 mg/kg body weight is administered. In one aspect, 2DG at about 5.0 mg/kg body weight to about 250 mg/kg body weight is administered. In one aspect, 2DG at about 10 mg/kg body weight to about 100 mg/kg body weight is administered. In one aspect, 2DG at about 25 mg/kg body weight to about 50 mg/kg body weight is administered. In one aspect, it is administered daily. According to Yamaguchi et al. (PLOS One, 2011, 6(9), e24102), 2DG accumulates predominantly in cancer cells compared to their normal counterpart cells or to other normal cells.

2-Deoxy-D-glucose is a glucose molecule which has the 2-hydroxyl group replaced by hydrogen. 2DG is transported across the plasma membrane by a glucose transporter. Once in the cytosol, 2DG is phosphorylated by hexokinase II and its product, 2-deoxyglucose 6-phosphate, is trapped in the cytosol and becomes an inhibitor of hexokinases, just as glucose becomes glucose 6-phosphate and becomes an inhibitor of hexokinases. However, as glucose 6-phosphate is hydrolyzed by glucose 6-phosphatase very rapidly, producing NADPH and generating energy, its counterpart, 2-deoxyglucose 6-phosphate, is a poorer substrate of glucose 6-phosphatase. Consequently, 2-deoxyglucose 6-phosphate accumulates in the cytosol, inhibiting hexokinases and lowering cellular energy levels. It is estimated that the intracellular half-life of 2-deoxyglucose 6-phosphate is approximately 50 minutes in cancer cells. Thus, 2DG acts as an inhibitor of the glycolytic pathway.

In one aspect, metformin is administered in combination with 2DG (Sahra et al., 2010).

In one embodiment, a pituitary adenoma that is sensitive to glucose limitation is treated with a glucose transporter (GLUT) inhibitor to induce infarction. As used herein, a “GLUT inhibitor” is an agent that inhibits SLC2A1 or SLC2A3 expression or activity. In one embodiment, a GLUT inhibitor selectively inhibits GLUT1, GLUT3, or both, as compared with inhibition of at least one other glucose transporter, preferably as compared with inhibition of multiple other glucose transporters. A selective GLUT inhibitor inhibits its target(s) (e.g., GLUT1 and/or GLUT3) with a lower IC₅₀ than non-target glucose transporters. In one embodiment, a GLUT inhibitor is a small molecule or polypeptide (e.g., an antibody) that binds to the GLUT1 or GLUT3 transporter and blocks the ability of the transporter to transport glucose. It would be appreciated by one of skill in the art based on the teachings herein that a non-human antibody may be used to generate a chimeric or humanized antibody, or a fully human antibody may be used. In some embodiments a GLUT inhibitor is a glucose analog such as 2-deoxyglucose. In one embodiment, a GLUT inhibitor is a flavonoid such as phloretin, genistein, or silybin/silibinin.

In one embodiment, the GLUT inhibitor is an siRNA that inhibits expression of SLC2A1 or SLC2A3. In one embodiment, a GLUT inhibitor is a glucose analog such as 2-deoxyglucose.

In one embodiment, the present invention provides compositions and methods for inducing infarction of a pituitary adenoma by inducing controlled hypoglycemia. In one aspect, insulin can be administered to induce controlled hypoglycemia, which in turn induces selective infarction of a pituitary adenoma. In one aspect, the insulin is long-lasting insulin. One of ordinary skill in the art can determine the dose of insulin to use based on various parameters of the diagnosis such as the size of the adenoma and the age, health, weight, and sex of the subject being treated. For example, in one aspect, insulin can be used at 0.15, 0.5, 1.0, 2.0, 5.0, 10.0, 15.0, at 20.0 units kg/body weight (Humulin, Eli Lilly, Indianapolis, Ind.). In one aspect, the insulin is administered intravenously (i.v.). In one aspect, is given i.v. over 90 seconds. In one aspect, it is given at a dose ranging from 0.10 IU/kg body weight to 10.0 IU/kg body weight. In one aspect, it is given at a dose ranging from 0.15 IU/kg body weight to 5.0 IU/kg body weight or about 5.5 IU to about 9.0 IU.

In one embodiment, the present invention provides compositions and methods for inducing infarction of a pituitary adenoma by methods of controlled systemic hypotension. In one aspect, this is brought about in stages to a mean arterial blood pressure (MAP) 30-40% below the patient's usual MAP but above 50 mm Hg. Various pharmacological agents can be used for inducing hypotension to treat pituitary adenomas. This can be accomplished with (a) deep anesthesia and heavy analgesia and (b) standard anesthesia and administration of hypotensive drugs. Potential hypotensive drugs include, but are not limited to sodium nitroprusside (SNP), nitroglycerin (NTG), trimethaphan, calcium channel antagonists (e.g., nicardipine), β-adrenoceptor antagonists (e.g., propranolol and esmolol), angiotensin converting enzyme (ACE) inhibitors, and adrenoceptor agonists (e.g., clonidine and dexmedetomidine). In one aspect, at least two agents are administered to a subject.

In one embodiment, the present invention provides compositions comprising at least one hypothalamic releasing factor for use in inducing infarction of a pituitary adenoma. Useful hypothalamic releasing factors include Thyrotropin-releasing hormone (TRH), Corticotropin-releasing hormone (CRH), Gonadotropin-releasing hormone (GnRH), and Growth hormone-releasing hormone (GHRH). In one embodiment, TRH is administered at a dose of about 200 micrograms (μg)/kg body weight. In one aspect, TRH is administered at a dose ranging from about 10 to about 500 μg/kg body weight. In one aspect, TRH is administered at a dose ranging from about 50 to about 400 μg/kg body weight. In one embodiment, CRH is administered at a dose of about 1.0 μg/kg body weight or a unit dose of about 100 μg. In one aspect, CRH is administered at a dose ranging from about 0.5 μg/kg body weight to about 100 μg/kg body weight. In one embodiment, GnRH is administered at a unit dose of about 100 μg. In one aspect, it is administered at a dose ranging from about 0.01 μg/kg body weight to about 5.0 μg/kg body weight. In one aspect, it is administered at a dose ranging from about 0.1 μg/kg body weight to about 1.0 μg/kg body weight. In embodiment, GHRH is administered at a dose of about 1.0 μg/kg body weight. In one embodiment, at least two hypothalamic releasing factors are administered.

In one aspect, a dose of the invention can be administered per day, per treatment, per cycle, or per procedure.

Depending on the dose given to a subject, it can also be administered more than once and when administered more than once the intervals can be varied and the dose and intervals can be determined by the physician. In one aspect, a dose can be broken up into smaller sub-unit doses.

In one embodiment, when the active ingredient needs to enter circulation and be delivered via blood, the active ingredient can be administered to achieve peak plasma concentrations of the active compound. This may be achieved, for example, by the intravenous injection of a 0.05 to 5% solution of the active ingredient, optionally in saline, or orally administered as a bolus containing the active ingredient.

Desirable blood levels may be maintained by continuous infusion to provide doses at a particular mg/kg/hr or by intermittent infusions containing a selected amount (mg/kg) of the active ingredient(s).

The desired dose may conveniently be presented in a single dose or as divided doses administered at appropriate intervals, for example, as two, three, four, or more sub-doses per day or per procedure. The sub-dose itself may be further divided, e.g., into a number of discrete loosely spaced administrations.

In one aspect, doses can be varied over time by either decreasing or increasing the dose in subsequent administrations. The method for what doses to use or vary can be determined, for example, by monitoring the subject and the pituitary tumor during treatment. In one aspect, the doses of an agent can vary per cycle of treatment. Depending on the particular agent or treatment, a cycle can vary. For example a cycle of treatment can be for multiple days or weeks at a time, including, but not limited to, cycles of 2, 3, 4, 5, 6, 7,8, 9, or 10 or more days or cycles of 1, 2, or 3 weeks. Doses can be administered, for example, daily, more than once per day, or every other day, depending on the treatment regimen that has been selected for that particular patient based on the diagnosis determined using the methods of the invention. For example, if a treatment regimen using 2DG is selected, a graded dosage can include an initial dose of 30 mg/kg body weight daily for 2 weeks, then 45 mg/kg, then 60 mg/kg, etc. Strategies for increasing doses include increasing each dose by a set percentage such as 33% or 50% relative to the first dose or the previous dose.

In one embodiment, the present invention provides for the use of 18F-Fluoro-2-deoxyglucose positron emission tomography/computed tomography (18F-FDG PET/CT) in subjects with pituitary adenoma for diagnosing, staging, detecting recurrent lesions, developing treatment regimens, and monitoring treatment response to the treatments disclosed herein.

The present invention further encompasses methods for determining a treatment regimen for a subject diagnosed with a pituitary adenoma.

Various aspects and embodiments of the invention are described in further detail below.

### BRIEF DESCRIPTION OF THE DRAWINGS

FIG. 1. Image from MRIs obtained 4 days after the onset of symptoms of pituitary apoplexy in a 57 year-old male (left, anteroposterior view). Repeat MRI 5 months later (right) shows no evidence of residual tumor.

FIG. 2, comprising four panels, depicts images from a 62 year old male with pituitary adenoma. This 62 year old male had a CT FDG-PET performed as a screening test. It revealed an incidentally discovered pituitary macroadenoma. Sagittal whole body view (Upper left) and axial cranial view of the FDG CT-PET (Upper right) show exuberant uptake of FDG in the small macroadenoma. Pituitary MRI after contrast shown in the antero-posterior (Lower left) and sagittal (Lower right) views. The arrow indicates the normal gland which has been displaced to the far left side of the sella by the tumor.

FIG. 3A-B. Sensitivity of pituitary adenoma cells to glucose deprivation. FIG. 3A—Pituitary tumor cells from a patient with Cushing's disease were isolated and exposed to increasing concentrations of glucose in the culture medium. For example, a control received no glucose and the treatment groups ranged from about 0.1 to 2.0 mg/ml glucose (0, 0.1, 0.2, 0.5, 1.0, 1.5, and 2.0). Normal blood glucose is 1 mg/ml (100 mg/dL). The tumor cells do not survive with acute deprivation of glucose. Viability was measured by a colorimetric cell proliferation assay (Aqueous One Proliferation Assay Solution (Promega, Madison, Wis.). Values are expressed as mean±SD, n=5 wells/condition). FIG. 3B. Two additional pituitary tumors were cultured (one growth hormone secreting tumor (left, n=4 wells/condition) and one non-secreting tumor (right, n=6 wells/condition) in the presence (black bar, 100 mg/dL) or absence (gray bar) of glucose. Normal human fibroblasts (Fib) (ATCC, Manassas, Va.) were included as a non-tumor control cell type (n=5 or 6 wells/condition). Pituitary tumor cells were sensitive to the absence of glucose, whereas the fibroblasts were not.

### DETAILED DESCRIPTION

Abbreviations and Acronyms

2DG—2-Deoxy-D-glucose

ACE—angiotensin converting enzyme

CNS—central nervous system

CRH—Corticotropin-releasing hormone

CT—computed tomography

FDG—fluorodeoxyglucose

Fib—fibroblast

g—gram

GHRH—Growth hormone-releasing hormone

GLUT—glucose transporter

GnRH—Gonadotropin-releasing hormone

IU—international unit

kg—kilogram

MAP—mean arterial blood pressure

μg—microgram

ml—milliliter

mm Hg—millimeters of mercury

MRI—magnetic resonance imaging

NTG—nitroglycerin

PET—positron emission tomography

SNP—sodium nitroprusside

TRH—Thyrotropin-releasing hormone

u—unit

VEGF—vascular endothelial growth factor

Definitions

In describing and claiming the invention, the following terminology will be used in accordance with the definitions set forth below.

The articles “a” and “an” are used herein to refer to one or to more than one (i.e., to at least one) of the grammatical object of the article. By way of example, “an element” means one element or more than one element.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. Therefore, about 50% means in the range of 45%-55%. Numerical ranges recited herein by endpoints include all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about.”

An “agent” of the invention as used and claimed herein refers to a drug or compound useful for treating a pituitary adenoma and inducing infarction of the adenoma.

As used herein, “an agent that controls systemic hypotension” refers to a drug, compound, or method used to induce and/or control hypotension.

As used herein, amino acids are represented by the full name thereof, by the three letter code corresponding thereto, or by the one-letter code corresponding thereto, as indicated in the following table:

| Full Name | Three-Letter Code | One-Letter Code |
|---|---|---|
| Aspartic Acid | Asp | D |
| Glutamic Acid | Glu | E |
| Lysine | Lys | K |
| Arginine | Arg | R |
| Histidine | His | H |
| Tyrosine | Tyr | Y |
| Cysteine | Cys | C |
| Asparagine | Asn | N |
| Glutamine | Gln | Q |
| Serine | Ser | S |
| Threonine | Thr | T |
| Glycine | Gly | G |
| Alanine | Ala | A |
| Valine | Val | V |
| Leucine | Leu | L |
| Isoleucine | Ile | I |
| Methionine | Met | M |
| Proline | Pro | P |
| Phenylalanine | Phe | F |
| Tryptophan | Trp | W |

The term “amino acid” as used herein is meant to include both natural and synthetic amino acids, and both D and L amino acids. “Standard amino acid” means any of the twenty standard L-amino acids commonly found in naturally occurring peptides. “Nonstandard amino acid residue” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions. Amino acids contained within the peptides of the present invention, and particularly at the carboxy- or amino-terminus, can be modified by methylation, amidation, acetylation or substitution with other chemical groups which can change the peptide's circulating half-life without adversely affecting their activity. Additionally, a disulfide linkage may be present or absent in the peptides of the invention.

The term “amino acid” is used interchangeably with “amino acid residue,” and may refer to a free amino acid and to an amino acid residue of a peptide. It will be apparent from the context in which the term is used whether it refers to a free amino acid or a residue of a peptide.

Amino acids have the following general structure:

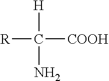

Amino acids may be classified into seven groups on the basis of the side chain R: (1) aliphatic side chains, (2) side chains containing a hydroxylic (OH) group, (3) side chains containing sulfur atoms, (4) side chains containing an acidic or amide group, (5) side chains containing a basic group, (6) side chains containing an aromatic ring, and (7) proline, an imino acid in which the side chain is fused to the amino group.

The nomenclature used to describe the peptide compounds of the present invention follows the conventional practice wherein the amino group is presented to the left and the carboxy group to the right of each amino acid residue. In the formulae representing selected specific embodiments of the present invention, the amino- and carboxy-terminal groups, although not specifically shown, will be understood to be in the form they would assume at physiologic pH values, unless otherwise specified.

The term “basic” or “positively charged” amino acid as used herein, refers to amino acids in which the R groups have a net positive charge at pH 7.0, and include, but are not limited to, the standard amino acids lysine, arginine, and histidine.

As used herein, an “analog” of a chemical compound is a compound that, by way of example, resembles another in structure but is not necessarily an isomer (e.g., 5-fluorouracil is an analog of thymine).

As used herein, the term “antisense oligonucleotide” or antisense nucleic acid means a nucleic acid polymer, at least a portion of which is complementary to a nucleic acid which is present in a normal cell or in an affected cell. “Antisense” refers particularly to the nucleic acid sequence of the non-coding strand of a double stranded DNA molecule encoding a protein, or to a sequence which is substantially homologous to the non-coding strand. As defined herein, an antisense sequence is complementary to the sequence of a double stranded DNA molecule encoding a protein. It is not necessary that the antisense sequence be complementary solely to the coding portion of the coding strand of the DNA molecule. The antisense sequence may be complementary to regulatory sequences specified on the coding strand of a DNA molecule encoding a protein, which regulatory sequences control expression of the coding sequences. The antisense oligonucleotides of the invention include, but are not limited to, phosphorothioate oligonucleotides and other modifications of oligonucleotides.

The term “binding” refers to the adherence of molecules to one another, such as, but not limited to, enzymes to substrates, ligands to receptors, antibodies to antigens, DNA binding domains of proteins to DNA, and DNA or RNA strands to complementary strands.

“Binding partner,” as used herein, refers to a molecule capable of binding to another molecule.

As used herein, the term “biologically active fragments” or “bioactive fragment” of the polypeptides encompasses natural or synthetic portions of the full-length protein that are capable of specific binding to their natural ligand or of performing the function of the protein.

The term “cancer”, as used herein, is defined as proliferation of cells whose unique trait—loss of normal controls—results in unregulated growth, lack of differentiation, local tissue invasion, and metastasis. Examples include but are not limited to, pituitary adenoma, melanoma, breast cancer, prostate cancer, ovarian cancer, uterine cancer, cervical cancer, skin cancer, pancreatic cancer, colorectal cancer, renal cancer and lung cancer.

As used herein, the term “carrier molecule” refers to any molecule that is chemically conjugated to the antigen of interest that enables an immune response resulting in antibodies specific to the native antigen.

The term “cell surface protein” means a protein found where at least part of the protein is exposed at the outer aspect of the cell membrane. Examples include growth factor receptors.

As used herein, the term “chemically conjugated,” or “conjugating chemically” refers to linking the antigen to the carrier molecule. This linking can occur on the genetic level using recombinant technology, wherein a hybrid protein may be produced containing the amino acid sequences, or portions thereof, of both the antigen and the carrier molecule. This hybrid protein is produced by an oligonucleotide sequence encoding both the antigen and the carrier molecule, or portions thereof. This linking also includes covalent bonds created between the antigen and the carrier protein using other chemical reactions, such as, but not limited to glutaraldehyde reactions. Covalent bonds may also be created using a third molecule bridging the antigen to the carrier molecule. These cross-linkers are able to react with groups, such as but not limited to, primary amines, sulfhydryls, carbonyls, carbohydrates or carboxylic acids, on the antigen and the carrier molecule. Chemical conjugation also includes non-covalent linkage between the antigen and the carrier molecule.

A “coding region” of a gene consists of the nucleotide residues of the coding strand of the gene and the nucleotides of the non-coding strand of the gene which are homologous with or complementary to, respectively, the coding region of an mRNA molecule which is produced by transcription of the gene.

The term “competitive sequence” refers to a peptide or a modification, fragment, derivative, or homolog thereof that competes with another peptide for its cognate binding site.

“Complementary” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. As used herein, the terms “complementary” or “complementarity” are used in reference to polynucleotides (i.e., a sequence of nucleotides) related by the base-pairing rules. For example, for the sequence “A-G-T,” is complementary to the sequence “T-C-A.”

Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

A “compound,” as used herein, refers to any type of substance or agent that is commonly considered a drug, or a candidate for use as a drug, as well as combinations and mixtures of the above.

As used herein, the term “conservative amino acid substitution” is defined herein as an amino acid exchange within one of the following five groups:

I. Small aliphatic, nonpolar or slightly polar residues:
Ala, Ser, Thr, Pro, Gly;

II. Polar, negatively charged residues and their amides:
Asp, Asn, Glu, Gln;

III. Polar, positively charged residues:
His, Arg, Lys;

IV. Large, aliphatic, nonpolar residues:
Met Leu, Ile, Val, Cys

V. Large, aromatic residues:
Phe, Tyr, Trp

As used herein, a “derivative” of a compound, when referring to a chemical compound, is one that may be produced from another compound of similar structure in one or more steps, as in replacement of H by an alkyl, acyl, or amino group.

The use of the word “detect” and its grammatical variants refers to measurement of the species without quantification, whereas use of the word “determine” or “measure” with their grammatical variants are meant to refer to measurement of the species with quantification. The terms “detect” and “identify” are used interchangeably herein.

As used herein, a “detectable marker” or a “reporter molecule” is an atom or a molecule that permits the specific detection of a compound comprising the marker in the presence of similar compounds without a marker. Detectable markers or reporter molecules include, e.g., radioactive isotopes, antigenic determinants, enzymes, nucleic acids available for hybridization, chromophores, fluorophores, chemiluminescent molecules, electrochemically detectable molecules, and molecules that provide for altered fluorescence-polarization or altered light-scattering.

As used herein, the term “domain” refers to a part of a molecule or structure that shares common physicochemical features, such as, but not limited to, hydrophobic, polar, globular and helical domains or properties such as ligand binding, signal transduction, cell penetration and the like. Specific examples of binding domains include, but are not limited to, DNA binding domains and ATP binding domains. As used herein, the term “effector domain” refers to a domain capable of directly interacting with an effector molecule, chemical, or structure in the cytoplasm, which is capable of regulating a biochemical pathway.

As used herein, an “effective amount” or “therapeutically effective amount” means an amount sufficient to produce a selected effect, such as alleviating symptoms of a disease or disorder. In the context of administering compounds in the form of a combination, such as multiple compounds, the amount of each compound, when administered in combination with another compound(s), may be different from when that compound is administered alone. Thus, an effective amount of a combination of compounds refers collectively to the combination as a whole, although the actual amounts of each compound may vary. The term “more effective” means that the selected effect is alleviated to a greater extent by one treatment relative to the second treatment to which it is being compared.

As used herein, an “essentially pure” preparation of a particular protein or peptide is a preparation wherein at least about 95%, and preferably at least about 99%, by weight, of the protein or peptide in the preparation is the particular protein or peptide.

As used in the specification and the appended claims, the terms “for example,” “for instance,” “such as,” “including” and the like are meant to introduce examples that further clarify more general subject matter. Unless otherwise specified, these examples are provided only as an aid for understanding the invention, and are not meant to be limiting in any fashion.

The terms “formula” and “structure” are used interchangeably herein.

As used herein the term “expression” when used in reference to a gene or protein, without further modification, is intended to encompass transcription of a gene and/or translation of the transcript into a protein.

A “fragment” or “segment” is a portion of an amino acid sequence, comprising at least one amino acid, or a portion of a nucleic acid sequence comprising at least one nucleotide. The terms “fragment” and “segment” are used interchangeably herein.

As used herein, the term “fragment,” as applied to a protein or peptide, can ordinarily be at least about 2-15 amino acids in length, at least about 15-25 amino acids, at least about 25-50 amino acids in length, at least about 50-75 amino acids in length, at least about 75-100 amino acids in length, and greater than 100 amino acids in length, depending on the particular protein or peptide being referred to.

As used herein, the term “fragment” as applied to a nucleic acid, may ordinarily be at least about 20 nucleotides in length, typically, at least about 50 nucleotides, more typically, from about 50 to about 100 nucleotides, preferably, at least about 100 to about 200 nucleotides, even more preferably, at least about 200 nucleotides to about 300 nucleotides, yet even more preferably, at least about 300 to about 350, even more preferably, at least about 350 nucleotides to about 500 nucleotides, yet even more preferably, at least about 500 to about 600, even more preferably, at least about 600 nucleotides to about 620 nucleotides, yet even more preferably, at least about 620 to about 650, and most preferably, the nucleic acid fragment will be greater than about 650 nucleotides in length.

As used herein, a “functional” molecule is a molecule in a form in which it exhibits a property or activity by which it is characterized. A functional enzyme, for example, is one that exhibits the characteristic catalytic activity by which the enzyme is characterized.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′ATTGCC5′ and 3′TATGGC share 50% homology.

As used herein, “homology” is used synonymously with “identity.”

The determination of percent identity between two nucleotide or amino acid sequences can be accomplished using a mathematical algorithm. For example, a mathematical algorithm useful for comparing two sequences is the algorithm of Karlin and Altschul (1990, Proc. Natl. Acad. Sci. USA 87:2264-2268), modified as in Karlin and Altschul (1993, Proc. Natl. Acad. Sci. USA 90:5873-5877). This algorithm is incorporated into the NBLAST and XBLAST programs of Altschul, et al. (1990, J. Mol. Biol. 215:403-410), and can be accessed, for example at the National Center for Biotechnology Information (NCBI) world wide web site. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al. (1997, Nucleic Acids Res. 25:3389-3402). Alternatively, PSI-Blast or PHI-Blast can be used to perform an iterated search which detects distant relationships between molecules (Id.) and relationships between molecules which share a common pattern. When utilizing BLAST, Gapped BLAST, PSI-Blast, and PHI-Blast programs, the default parameters of the respective programs (e.g., XBLAST and NBLAST) can be used.

The percent identity between two sequences can be determined using techniques similar to those described above, with or without allowing gaps. In calculating percent identity, typically exact matches are counted.

As used herein, the term “hybridization” is used in reference to the pairing of complementary nucleic acids. Hybridization and the strength of hybridization (i.e., the strength of the association between the nucleic acids) is impacted by such factors as the degree of complementarity between the nucleic acids, stringency of the conditions involved, the length of the formed hybrid, and the G:C ratio within the nucleic acids.

The term “inhibit,” as used herein, refers to the ability of a compound, agent, or method to reduce or impede a described function, level, activity, rate, etc., based on the context in which the term “inhibit” is used. Preferably, inhibition is by at least 10%, more preferably by at least 25%, even more preferably by at least 50%, and most preferably, the function is inhibited by at least 75%. The term “inhibit” is used interchangeably with “reduce” and “block.”

The term “inhibit a protein,” as used herein, refers to any method or technique which inhibits protein synthesis, levels, activity, or function, as well as methods of inhibiting the induction or stimulation of synthesis, levels, activity, or function of the protein of interest. The term also refers to any metabolic or regulatory pathway which can regulate the synthesis, levels, activity, or function of the protein of interest. The term includes binding with other molecules and complex formation. Therefore, the term “protein inhibitor” refers to any agent or compound, the application of which results in the inhibition of protein function or protein pathway function. However, the term does not imply that each and every one of these functions must be inhibited at the same time.

As used herein “injecting or applying” includes administration of a compound of the invention by any number of routes and means including, but not limited to, topical, oral, buccal, intravenous, intramuscular, intra arterial, intramedullary, intrathecal, intraventricular, transdermal, subcutaneous, intraperitoneal, intranasal, enteral, topical, sublingual, vaginal, ophthalmic, pulmonary, or rectal means.

As used herein, an “instructional material” includes a publication, a recording, a diagram, or any other medium of expression which can be used to communicate the usefulness of the peptide of the invention in the kit. The instructional material of the kit of the invention may, for example, be affixed to a container which contains the identified compound(s) invention or be shipped together with a container which contains the identified compound. Alternatively, the instructional material may be shipped separately from the container with the intention that the instructional material and the compound be used cooperatively by the recipient.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence.

The terms “kg body weight” or “per kilogram body weight” refer to how a dose of an agent of the invention is administered based on the weight of the subject to whom it is administered.

A “ligand” is a compound that specifically binds to a target receptor.

A “receptor” is a compound that specifically binds to a ligand.

As used herein, the term “linkage” refers to a connection between two groups. The connection can be either covalent or non-covalent, including but not limited to ionic bonds, hydrogen bonding, and hydrophobic/hydrophilic interactions.

As used herein, the term “linker” refers to a molecule that joins two other molecules either covalently or noncovalently, e.g., through ionic or hydrogen bonds or van der Waals interactions.

The term “measuring the level of expression” or “determining the level of expression” as used herein refers to any measure or assay which can be used to correlate the results of the assay with the level of expression of a gene or protein of interest. Such assays include measuring the level of mRNA, protein levels, etc., and can be performed by assays such as northern and western blot analyses, binding assays, immunoblots, etc. The level of expression can include rates of expression and can be measured in terms of the actual amount of an mRNA or protein present. Such assays are coupled with processes or systems to store and process information and to help quantify levels, signals, etc. and to digitize the information for use in comparing levels.

The term “modulate”, as used herein, refers to changing the level of an activity, function, or process. The term “modulate” encompasses both inhibiting and stimulating an activity, function, or process.

The term “nasal administration” in all its grammatical forms refers to administration of at least one compound of the invention through the nasal mucous membrane to the bloodstream for systemic delivery of at least one compound of the invention. The advantages of nasal administration for delivery are that it does not require injection using a syringe and needle, it avoids necrosis that can accompany intramuscular administration of drugs, and trans-mucosal administration of a drug is highly amenable to self administration.

As used herein, the term “nucleic acid” encompasses RNA as well as single and double-stranded DNA and cDNA. Furthermore, the terms, “nucleic acid,” “DNA,” “RNA” and similar terms also include nucleic acid analogs, i.e. analogs having other than a phosphodiester backbone. For example, the so-called “peptide nucleic acids,” which are known in the art and have peptide bonds instead of phosphodiester bonds in the backbone, are considered within the scope of the present invention. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil). Conventional notation is used herein to describe polynucleotide sequences: the left-hand end of a single-stranded polynucleotide sequence is the 5′-end; the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences.”

The term “Oligonucleotide” typically refers to short polynucleotides, generally no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

“Operably linked” refers to a juxtaposition wherein the components are configured so as to perform their usual function. Thus, control sequences or promoters operably linked to a coding sequence are capable of effecting the expression of the coding sequence. By describing two polynucleotides as “operably linked” is meant that a single-stranded or double-stranded nucleic acid moiety comprises the two polynucleotides arranged within the nucleic acid moiety in such a manner that at least one of the two polynucleotides is able to exert a physiological effect by which it is characterized upon the other. By way of example, a promoter operably linked to the coding region of a gene is able to promote transcription of the coding region.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

The term “peptide” typically refers to short polypeptides.

The term “per application” as used herein refers to administration of a compositions, drug, or compound to a subject.

The term “pharmaceutical composition” shall mean a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

As used herein, the term “pharmaceutically-acceptable carrier” means a chemical composition with which an appropriate compound or derivative can be combined and which, following the combination, can be used to administer the appropriate compound to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

“Pharmaceutically acceptable” means physiologically tolerable, for either human or veterinary application.

As used herein, “pharmaceutical compositions” include formulations for human and veterinary use.

“Plurality” means at least two.

A “polynucleotide” means a single strand or parallel and anti-parallel strands of a nucleic acid. Thus, a polynucleotide may be either a single-stranded or a double-stranded nucleic acid.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof.

By “purified bacterial components” is meant proteins purified from bacteria or purified proteins made using bacterial protein sequences.

By “synthesis in vitro” is meant cellulose synthesis that is not occurring in a cell, although it does not exclude synthesis where cellular components are added or the use of cells that either do not have their own endogenous cellulose synthetic machinery or cells that no longer have such machinery.

“Synthetic peptides or polypeptides” means a non-naturally occurring peptide or polypeptide. Synthetic peptides or polypeptides can be synthesized, for example, using an automated polypeptide synthesizer. Various solid phase peptide synthesis methods are known to those of skill in the art.

As used herein, “protecting group” with respect to a terminal amino group refers to a terminal amino group of a peptide, which terminal amino group is coupled with any of various amino-terminal protecting groups traditionally employed in peptide synthesis. Such protecting groups include, for example, acyl protecting groups such as formyl, acetyl, benzoyl, trifluoroacetyl, succinyl, and methoxysuccinyl; aromatic urethane protecting groups such as benzyloxycarbonyl; and aliphatic urethane protecting groups, for example, tert-butoxycarbonyl or adamantyloxycarbonyl. See Gross and Mienhofer, eds., The Peptides, vol. 3, pp. 3-88 (Academic Press, New York, 1981) for suitable protecting groups.

As used herein, “protecting group” with respect to a terminal carboxy group refers to a terminal carboxyl group of a peptide, which terminal carboxyl group is coupled with any of various carboxyl-terminal protecting groups. Such protecting groups include, for example, tert-butyl, benzyl or other acceptable groups linked to the terminal carboxyl group through an ester or ether bond.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process. A “highly purified” compound as used herein refers to a compound that is greater than 90% pure. In particular, purified sperm cell DNA refers to DNA that does not produce significant detectable levels of non-sperm cell DNA upon PCR amplification of the purified sperm cell DNA and subsequent analysis of that amplified DNA. A “significant detectable level” is an amount of contaminate that would be visible in the presented data and would need to be addressed/explained during analysis of the forensic evidence.

The term “protein regulatory pathway”, as used herein, refers to both the upstream regulatory pathway which regulates a protein, as well as the downstream events which that protein regulates. Such regulation includes, but is not limited to, transcription, translation, levels, activity, posttranslational modification, and function of the protein of interest, as well as the downstream events which the protein regulates. The terms “protein pathway” and “protein regulatory pathway” are used interchangeably herein.

A “recombinant polypeptide” is one which is produced upon expression of a recombinant polynucleotide.

The term “regulate” refers to either stimulating or inhibiting a function or activity of interest.

A “sample,” as used herein, refers preferably to a biological sample from a subject, including, but not limited to, normal tissue samples, diseased tissue samples, biopsies, blood, saliva, feces, semen, tears, and urine. A sample can also be any other source of material obtained from a subject which contains cells, tissues, or fluid of interest. A sample can also be obtained from cell or tissue culture.

By “small interfering RNAs (siRNAs)” is meant, inter alia, an isolated dsRNA molecule comprised of both a sense and an anti-sense strand. In one aspect, it is greater than 10 nucleotides in length. siRNA also refers to a single transcript which has both the sense and complementary antisense sequences from the target gene, e.g., a hairpin. siRNA further includes any form of dsRNA (proteolytically cleaved products of larger dsRNA, partially purified RNA, essentially pure RNA, synthetic RNA, recombinantly produced RNA) as well as altered RNA that differs from naturally occurring RNA by the addition, deletion, substitution, and/or alteration of one or more nucleotides.

By the term “specifically binds to”, as used herein, is meant when a compound or ligand functions in a binding reaction or assay conditions which is determinative of the presence of the compound in a sample of heterogeneous compounds.

The term “standard,” as used herein, refers to something used for comparison. For example, it can be a known standard agent or compound which is administered and used for comparing results when administering a test compound, or it can be a standard parameter or function which is measured to obtain a control value when measuring an effect of an agent or compound on a parameter or function. Standard can also refer to an “internal standard”, such as an agent or compound which is added at known amounts to a sample and is useful in determining such things as purification or recovery rates when a sample is processed or subjected to purification or extraction procedures before a marker of interest is measured. Internal standards are often a purified marker of interest which has been labeled, such as with a radioactive isotope, allowing it to be distinguished from an endogenous marker.

A “subject” of analysis, diagnosis, or treatment is an animal. Such animals include mammals, preferably a human.

As used herein, a “subject in need thereof” is a patient, animal, mammal, or human, who will benefit from the method of this invention.

As used herein, a “substantially homologous amino acid sequences” includes those amino acid sequences which have at least about 95% homology, preferably at least about 96% homology, more preferably at least about 97% homology, even more preferably at least about 98% homology, and most preferably at least about 99% or more homology to an amino acid sequence of a reference antibody chain. Amino acid sequence similarity or identity can be computed by using the BLASTP and TBLASTN programs which employ the BLAST (basic local alignment search tool) 2.0.14 algorithm. The default settings used for these programs are suitable for identifying substantially similar amino acid sequences for purposes of the present invention.

The term “substantially pure” describes a compound, e.g., a protein or polypeptide which has been separated from components which naturally accompany it. Typically, a compound is substantially pure when at least 10%, more preferably at least 20%, more preferably at least 50%, more preferably at least 60%, more preferably at least 75%, more preferably at least 90%, and most preferably at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis, or HPLC analysis. A compound, e.g., a protein, is also substantially purified when it is essentially free of naturally associated components or when it is separated from the native contaminants which accompany it in its natural state.

The term “symptom,” as used herein, refers to any morbid phenomenon or departure from the normal in structure, function, or sensation, experienced by the patient and indicative of disease. In contrast, a “sign” is objective evidence of disease. For example, a bloody nose is a sign. It is evident to the patient, doctor, nurse and other observers.

A “therapeutic” treatment is a treatment administered to a subject who exhibits signs of pathology for the purpose of diminishing or eliminating those signs.

A “therapeutically effective amount” of a compound is that amount of compound which is sufficient to provide a beneficial effect to the subject to which the compound is administered.

The term to “treat,” as used herein, means reducing the frequency with which symptoms are experienced by a patient or subject or administering an agent or compound to reduce the frequency with which symptoms are experienced.

A “prophylactic” treatment is a treatment administered to a subject who does not exhibit signs of a disease or exhibits only early signs of the disease for the purpose of decreasing the risk of developing pathology associated with the disease.

As used herein, a “unit dose” is a discrete amount of the pharmaceutical composition comprising a predetermined amount of the active ingredient.

### Embodiments

Very few pituitary tumors are malignant. Pituitary tumors can be divided into three groups:

Benign pituitary adenomas: Tumors that are not cancer. These tumors grow very slowly and do not spread from the pituitary gland to other parts of the body.

Invasive pituitary adenomas: Benign tumors that may spread to bones of the skull or the sinus cavity below the pituitary gland.

Pituitary carcinomas: Tumors that are malignant (cancer). These pituitary tumors spread into other areas of the central nervous system (brain and spinal cord) or outside of the central nervous system.

Pituitary tumors may be either non-functioning or functioning. Non-functioning pituitary tumors do not make hormones. Functioning pituitary tumors make more than the normal amount of one or more hormones. Most pituitary tumors are functioning tumors. The extra hormones made by pituitary tumors may cause certain signs or symptoms of disease. There are multiple types of adenomas, classified by size and whether they produce hormones.

The most common symptoms of adenomas include headaches, vision problems that cannot be easily explained, menstrual cycle changes in women, mood swings or behavior changes, erectile dysfunction, and weight change. Blood and urine tests to measure hormone levels and medical imaging provide the best means of diagnosing pituitary tumors. Diagnostic imaging may include a high-resolution, T1 weighted, gadolinium enhanced MRI. In addition, blood and urine tests to obtain endocrine diagnostics may be performed to establish basal levels of PRL, GH, IGF-1, free thyroxine, cortisol, and testosterone (in males) levels.

Specific treatment for adenomas is generally coordinated by a neurosurgeon and an endocrinologist. Treatment may include surgery, including surgical removal via a procedure called endonasal transphenoidal endoscopic surgery, medical therapy, radiation therapy, hormone therapy, and/or observation.

Insulin can be administered to induce controlled hypoglycemia, which in turn induces selective infarction of a pituitary adenoma. One of ordinary skill in the art can determine the dose of insulin to use based on various parameters such as the size of the adenoma and the age, health, and sex of the subject being treated. For example, in one aspect, insulin can be used at 0.15, 0.5, 1.0, 2.0, 5.0, 10.0, 15.0, at 20.0 units kg/ml (Humulin, Eli Lilly, Indianapolis, Ind.). In one aspect, the insulin is administered intravenously. In one aspect, is given i.v. over 90 seconds. In one aspect, it is given at a dose ranging from 0.10 IU/kg body weight to 10.0 IU/kg body weight. In one aspect, it is given at a dose ranging from 0.15 IU/kg body weight to 5.0 IU/kg body weight or about 5.5 IU to about 9.0 IU.

Antibodies and other peptides can be conjugated to a number of agents capable of being imaged in vivo and used for imaging/detection in ex vivo tests and assays such as immunofluorescence, ELISA, etc. In one embodiment, the antibody is detected using at least one of enzyme-linked immunoassay, western blot, lateral flow membrane test, latex agglutination, and other forms of immunochromatography or immunoassay utilizing at least one antibody.

Multiple techniques for measuring proteins and peptides are known in the art or described herein and can use in the practice of the invention. These include, but are not limited to, for example:

Electrochemiluminescent immunoassay;

Bioluminescent Immunoassay (for example, with use of apoaequorin and oelenterazine);

Luminescent oxygen channeling immunoassay (LOCI);

The Erenna Immunoassay System (a modified microparticle-based sandwich immunoassay with single-molecule counting);

Nanoparticle Immunoassay: nano-particles, spheres, or tubes as solid phases
upconverting phosphor nanoparticle using antiStokes shift
quantum dot immunoassay (Heterogeneous immunoassay in which a nanometer-sized (less than 10 nm) semiconductor quantum dot is used as a label. A quantum dot is a highly fluorescent nanocrystal composed of CdSe, CdS, ZnSe, InP, or InAs or a layer of ZnS or CdS on, for example, a CdSe core);

Fluorescence Excitation Transfer Immunoassay;

ImmunoPCR Immunoassay;

Solid Phase, Light-Scattering Immunoassay: Indium spheres are coated on glass to measure an antibody binding to an antigen. Binding of antibodies to antigens increases dielectric layer thickness, which produces a greater degree of scatter than in areas where only an antigen is bound. Quantitation is achieved by densitometry; and

Surface Effect Immunoassay: with antibody immobilized on the surface of a waveguide (a quartz, glass, or plastic slide, or a gold- or silver-coated prism), and binding of antigen measured directly by total internal reflection fluorescence, surface plasmon resonance, or attenuated total reflection.

In one aspect, an antibody or a fragment or homolog thereof of the invention can be conjugated to an imaging agent. In one embodiment, antibody complex comprises an imaging agent selected from the group consisting of a radionuclide, a radiological contrast agent, a paramagnetic ion, a metal, a biological tag, a fluorescent label, a chemiluminescent label, an ultrasound contrast agent and a photoactive agent. In one aspect, the imaging agent is a radionuclide. In one aspect, the radionuclide is selected from the group consisting of ¹¹⁰In, ¹¹¹In, ¹⁷⁷Lu, ¹⁸F, ⁵²Fe, ⁶²Cu, ⁶⁴Cu, ⁶⁷Cu, ⁶⁷Ga, ⁶⁸Ga, ⁸⁶Y, ⁹⁰Y, ⁸⁹Zr, ^{94m}Tc, ⁹⁴Tc, ^{99m}Tc, ¹²⁰I, ¹²³I, ¹²⁴I, ¹²⁵I, ¹³¹I, ¹⁵⁴⁻¹⁵⁸Gd, ³²P, ¹¹C, ¹³N, ¹⁵O, ¹⁸⁶Re, ¹⁸⁸Re, ⁵¹Mn, ⁵²mMn, ⁵⁵Co, ⁷²As, ⁷⁵Br, ⁷⁶Br, ⁸²mRb, ⁸³Sr, and other gamma-, beta-, or positron-emitters. In one aspect, the radionuclide is ¹¹¹In.

The invention further provides for use of the monoclonal antibodies described herein for drug delivery and for diagnostics. For example, various agents as described herein can be conjugated to the antibodies. Drugs such as calicheamicin, peptides such as D(KLAKLAK)², and radionuclides such as beta ⁹⁰Y, gamma ¹³¹I, and positron ¹²⁴I emitters can be conjugated to monoclonal antibodies.

Pharmaceutical Compositions and Administration

The present invention is also directed to pharmaceutical compositions comprising the compounds of the present invention. More particularly, such compounds can be formulated as pharmaceutical compositions using standard pharmaceutically acceptable carriers, fillers, solublizing agents and stabilizers known to those skilled in the art.

The invention is also directed to methods of administering the compounds of the invention to a subject. In one embodiment, the invention provides a method of treating a subject by administering compounds identified using the methods of the invention description. Pharmaceutical compositions comprising the present compounds are administered to a subject in need thereof by any number of routes including, but not limited to, topical, oral, intravenous, intramuscular, intra-arterial, intramedullary, intrathecal, intraventricular, transdermal, subcutaneous, intraperitoneal, intranasal, enteral, topical, sublingual, or rectal means.

In accordance with one embodiment, a method of treating a subject in need of such treatment is provided. The method comprises administering a pharmaceutical composition comprising at least one compound of the present invention to a subject in need thereof. Compounds identified by the methods of the invention can be administered with known compounds or other medications as well.

The invention also encompasses the use of pharmaceutical compositions of an appropriate compound, and homologs, fragments, analogs, or derivatives thereof to practice the methods of the invention, the composition comprising at least one appropriate compound, and homolog, fragment, analog, or derivative thereof and a pharmaceutically-acceptable carrier.

The pharmaceutical compositions useful for practicing the invention may be administered to deliver a dose of between 1 ng/kg/day and 100 mg/kg/day.

The invention encompasses the preparation and use of pharmaceutical compositions comprising a compound useful for treatment of the diseases disclosed herein as an active ingredient. Such a pharmaceutical composition may consist of the active ingredient alone, in a form suitable for administration to a subject, or the pharmaceutical composition may comprise the active ingredient and one or more pharmaceutically acceptable carriers, one or more additional ingredients, or some combination of these. The active ingredient may be present in the pharmaceutical composition in the form of a physiologically acceptable ester or salt, such as in combination with a physiologically acceptable cation or anion, as is well known in the art.

As used herein, the term “physiologically acceptable” ester or salt means an ester or salt form of the active ingredient which is compatible with any other ingredients of the pharmaceutical composition, which is not deleterious to the subject to which the composition is to be administered.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

It will be understood by the skilled artisan that such pharmaceutical compositions are generally suitable for administration to animals of all sorts. Subjects to which administration of the pharmaceutical compositions of the invention is contemplated include, but are not limited to, humans and other primates, mammals including commercially relevant mammals such as cattle, pigs, horses, sheep, cats, and dogs, birds including commercially relevant birds such as chickens, ducks, geese, and turkeys. The invention is also contemplated for use in contraception for nuisance animals such as rodents.

A pharmaceutical composition of the invention may be prepared, packaged, or sold in bulk, as a single unit dose, or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents. Particularly contemplated additional agents include anti-emetics and scavengers such as cyanide and cyanate scavengers.

Controlled- or sustained-release formulations of a pharmaceutical composition of the invention may be made using conventional technology.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed., 1985, Remington's Pharmaceutical Sciences, Mack Publishing Co., Easton, Pa., which is incorporated herein by reference.

Typically, dosages of the compound of the invention which may be administered to an animal, preferably a human, range in amount from 1 μg to about 100 g per kilogram of body weight of the animal. While the precise dosage administered will vary depending upon any number of factors, including but not limited to, the type of animal and type of disease state being treated, the age of the animal and the route of administration. Preferably, the dosage of the compound will vary from about 1 mg to about 10 g per kilogram of body weight of the animal. More preferably, the dosage will vary from about 10 mg to about 1 g per kilogram of body weight of the animal.

The compound may be administered to an animal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the condition or disease being treated, the type and age of the animal, etc.

Suitable preparations of vaccines include injectables, either as liquid solutions or suspensions, however, solid forms suitable for solution in, suspension in, liquid prior to injection, may also be prepared. The preparation may also be emulsified, or the polypeptides encapsulated in liposomes. The active immunogenic ingredients are often mixed with excipients which are pharmaceutically acceptable and compatible with the active ingredient. Suitable excipients are, for example, water saline, dextrose, glycerol, ethanol, or the like and combinations thereof. In addition, if desired, the vaccine preparation may also include minor amounts of auxiliary substances such as wetting or emulsifying agents, pH buffering agents, and/or adjuvants which enhance the effectiveness of the vaccine.

The invention is also directed to methods of administering the compounds of the invention to a subject. In one embodiment, the invention provides a method of treating a subject by administering compounds identified using the methods of the invention. Pharmaceutical compositions comprising the present compounds are administered to an individual in need thereof by any number of routes including, but not limited to, topical, oral, intravenous, intramuscular, intra arterial, intramedullary, intrathecal, intraventricular, transdermal, subcutaneous, intraperitoneal, intranasal, enteral, topical, sublingual, or rectal means.

In accordance with one embodiment, a method of treating and vaccinating a subject in need of such treatment is provided. The method comprises administering a pharmaceutical composition comprising at least one compound of the present invention to a subject in need thereof. Compounds identified by the methods of the invention can be administered with known compounds or other medications as well.

For oral administration, the active ingredient can be administered in solid dosage forms, such as capsules, tablets, and powders, or in liquid dosage forms, such as elixirs, syrups, and suspensions. Active component(s) can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate, and the like. Examples of additional inactive ingredients that may be added to provide desirable color, taste, stability, buffering capacity, dispersion or other known desirable features are red iron oxide, silica gel, sodium lauryl sulfate, titanium dioxide, edible white ink and the like. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

The invention also includes a kit comprising the composition of the invention and an instructional material which describes adventitially administering the composition to a cell or a tissue of a mammal. In another embodiment, this kit comprises a (preferably sterile) solvent suitable for dissolving or suspending the composition of the invention prior to administering the compound to the mammal.

As used herein, an “instructional material” includes a publication, a recording, a diagram, or any other medium of expression which can be used to communicate the usefulness of the peptide of the invention in the kit for effecting alleviation of the various diseases or disorders recited herein. Optionally, or alternately, the instructional material may describe one or more methods of alleviation the diseases or disorders in a cell or a tissue of a mammal. The instructional material of the kit of the invention may, for example, be affixed to a container which contains the peptide of the invention or be shipped together with a container which contains the peptide. Alternatively, the instructional material may be shipped separately from the container with the intention that the instructional material and the compound be used cooperatively by the recipient.

Other techniques known in the art may be used in the practice of the present invention, including those described in international patent application WO 2006/091535 (PCT/US2006/005970), the entirety of which is incorporated by reference herein.

Peptide Modification and Preparation

Peptide preparation is described in the Examples. It will be appreciated, of course, that the proteins or peptides of the invention may incorporate amino acid residues which are modified without affecting activity. For example, the termini may be derivatized to include blocking groups, i.e. chemical substituents suitable to protect and/or stabilize the N- and C-termini from “undesirable degradation”, a term meant to encompass any type of enzymatic, chemical or biochemical breakdown of the compound at its termini which is likely to affect the function of the compound, i.e. sequential degradation of the compound at a terminal end thereof.

Blocking groups include protecting groups conventionally used in the art of peptide chemistry which will not adversely affect the in vivo activities of the peptide. For example, suitable N-terminal blocking groups can be introduced by alkylation or acylation of the N-terminus. Examples of suitable N-terminal blocking groups include C₁-C₅ branched or unbranched alkyl groups, acyl groups such as formyl and acetyl groups, as well as substituted forms thereof, such as the acetamidomethyl (Acm) group. Desamino analogs of amino acids are also useful N-terminal blocking groups, and can either be coupled to the N-terminus of the peptide or used in place of the N-terminal reside. Suitable C-terminal blocking groups, in which the carboxyl group of the C-terminus is either incorporated or not, include esters, ketones or amides. Ester or ketone-forming alkyl groups, particularly lower alkyl groups such as methyl, ethyl and propyl, and amide-forming amino groups such as primary amines (—NH₂), and mono- and di-alkylamino groups such as methylamino, ethylamino, dimethylamino, diethylamino, methylethylamino and the like are examples of C-terminal blocking groups. Descarboxylated amino acid analogues such as agmatine are also useful C-terminal blocking groups and can be either coupled to the peptide's C-terminal residue or used in place of it. Further, it will be appreciated that the free amino and carboxyl groups at the termini can be removed altogether from the peptide to yield desamino and descarboxylated forms thereof without affect on peptide activity.

Acid addition salts of the present invention are also contemplated as functional equivalents. Thus, a peptide in accordance with the present invention treated with an inorganic acid such as hydrochloric, hydrobromic, sulfuric, nitric, phosphoric, and the like, or an organic acid such as an acetic, propionic, glycolic, pyruvic, oxalic, malic, malonic, succinic, maleic, fumaric, tataric, citric, benzoic, cinnamie, mandelic, methanesulfonic, ethanesulfonic, p-toluenesulfonic, salicyclic and the like, to provide a water soluble salt of the peptide is suitable for use in the invention.

Modifications (which do not normally alter primary sequence) include in vivo, or in vitro chemical derivatization of polypeptides, e.g., acetylation, or carboxylation. Also included are modifications of glycosylation, e.g., those made by modifying the glycosylation patterns of a polypeptide during its synthesis and processing or in further processing steps; e.g., by exposing the polypeptide to enzymes which affect glycosylation, e.g., mammalian glycosylating or deglycosylating enzymes. Also embraced are sequences which have phosphorylated amino acid residues, e.g., phosphotyrosine, phosphoserine, or phosphothreonine.

Also included are polypeptides which have been modified using ordinary molecular biological techniques so as to improve their resistance to proteolytic degradation or to optimize solubility properties or to render them more suitable as a therapeutic agent. Analogs of such polypeptides include those containing residues other than naturally occurring L-amino acids, e.g., D-amino acids or non-naturally occurring or non-standard synthetic amino acids. The peptides of the invention are not limited to products of any of the specific exemplary processes listed herein.

The invention includes the use of beta-alanine (also referred to as β-alanine, β-Ala, bA, and βA, having the structure:

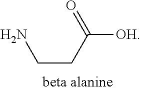

Sequences are provided herein which use the symbol “βA”, but in the Sequence Listing submitted herewith “βA” is provided as “Xaa” and reference in the text of the Sequence Listing indicates that Xaa is beta alanine.

Peptides useful in the present invention, such as standards, or modifications for analysis, may be readily prepared by standard, well-established techniques, such as solid-phase peptide synthesis (SPPS) as described by Stewart et al. in Solid Phase Peptide Synthesis, 2nd Edition, 1984, Pierce Chemical Company, Rockford, Ill.; and as described by Bodanszky and Bodanszky in The Practice of Peptide Synthesis, 1984, Springer-Verlag, New York. At the outset, a suitably protected amino acid residue is attached through its carboxyl group to a derivatized, insoluble polymeric support, such as cross-linked polystyrene or polyamide resin. “Suitably protected” refers to the presence of protecting groups on both the α-amino group of the amino acid, and on any side chain functional groups. Side chain protecting groups are generally stable to the solvents, reagents and reaction conditions used throughout the synthesis, and are removable under conditions which will not affect the final peptide product. Stepwise synthesis of the oligopeptide is carried out by the removal of the N-protecting group from the initial amino acid, and couple thereto of the carboxyl end of the next amino acid in the sequence of the desired peptide. This amino acid is also suitably protected. The carboxyl of the incoming amino acid can be activated to react with the N-terminus of the support-bound amino acid by formation into a reactive group such as formation into a carbodiimide, a symmetric acid anhydride or an “active ester” group such as hydroxybenzotriazole or pentafluorophenly esters.

Examples of solid phase peptide synthesis methods include the BOC method which utilized tert-butyloxcarbonyl as the α-amino protecting group, and the FMOC method which utilizes 9-fluorenylmethyloxcarbonyl to protect the α-amino of the amino acid residues, both methods of which are well-known by those of skill in the art.

Incorporation of N- and/or C-blocking groups can also be achieved using protocols conventional to solid phase peptide synthesis methods. For incorporation of C-terminal blocking groups, for example, synthesis of the desired peptide is typically performed using, as solid phase, a supporting resin that has been chemically modified so that cleavage from the resin results in a peptide having the desired C-terminal blocking group. To provide peptides in which the C-terminus bears a primary amino blocking group, for instance, synthesis is performed using a p-methylbenzhydrylamine (MBHA) resin so that, when peptide synthesis is completed, treatment with hydrofluoric acid releases the desired C-terminally amidated peptide. Similarly, incorporation of an N-methylamine blocking group at the C-terminus is achieved using N-methylaminoethyl-derivatized DVB, resin, which upon HF treatment releases a peptide bearing an N-methylamidated C-terminus. Blockage of the C-terminus by esterification can also be achieved using conventional procedures. This entails use of resin/blocking group combination that permits release of side-chain peptide from the resin, to allow for subsequent reaction with the desired alcohol, to form the ester function. FMOC protecting group, in combination with DVB resin derivatized with methoxyalkoxybenzyl alcohol or equivalent linker, can be used for this purpose, with cleavage from the support being effected by TFA in dicholoromethane. Esterification of the suitably activated carboxyl function e.g. with DCC, can then proceed by addition of the desired alcohol, followed by deprotection and isolation of the esterified peptide product.

Incorporation of N-terminal blocking groups can be achieved while the synthesized peptide is still attached to the resin, for instance by treatment with a suitable anhydride and nitrile. To incorporate an acetyl blocking group at the N-terminus, for instance, the resin-coupled peptide can be treated with 20% acetic anhydride in acetonitrile. The N-blocked peptide product can then be cleaved from the resin, deprotected and subsequently isolated.

To ensure that the peptide obtained from either chemical or biological synthetic techniques is the desired peptide, analysis of the peptide composition should be conducted. Such amino acid composition analysis may be conducted using high resolution mass spectrometry to determine the molecular weight of the peptide. Alternatively, or additionally, the amino acid content of the peptide can be confirmed by hydrolyzing the peptide in aqueous acid, and separating, identifying and quantifying the components of the mixture using HPLC, or an amino acid analyzer. Protein sequenators, which sequentially degrade the peptide and identify the amino acids in order, may also be used to determine definitely the sequence of the peptide.

Prior to its use, the peptide may be purified to remove contaminants. In this regard, it will be appreciated that the peptide will be purified so as to meet the standards set out by the appropriate regulatory agencies. Any one of a number of a conventional purification procedures may be used to attain the required level of purity including, for example, reversed-phase high performance liquid chromatography (HPLC) using an alkylated silica column such as C₄-, C₈- or C₁₈-silica. A gradient mobile phase of increasing organic content is generally used to achieve purification, for example, acetonitrile in an aqueous buffer, usually containing a small amount of trifluoroacetic acid. Ion-exchange chromatography can be also used to separate peptides based on their charge.

Substantially pure protein obtained as described herein may be purified by following known procedures for protein purification, wherein an immunological, enzymatic or other assay is used to monitor purification at each stage in the procedure. Protein purification methods are well known in the art, and are described, for example in Deutscher et al. (ed., 1990, Guide to Protein Purification, Harcourt Brace Jovanovich, San Diego).

As discussed, modifications or optimizations of peptide ligands of the invention are within the scope of the application. Modified or optimized peptides are included within the definition of peptide binding ligand. Specifically, a peptide sequence identified can be modified to optimize its potency, pharmacokinetic behavior, stability and/or other biological, physical and chemical properties.

Amino Acid Substitutions

In certain embodiments, the disclosed methods and compositions may involve preparing peptides with one or more substituted amino acid residues.

In various embodiments, the structural, physical and/or therapeutic characteristics of peptide sequences may be optimized by replacing one or more amino acid residues.

Other modifications can also be incorporated without adversely affecting the activity and these include, but are not limited to, substitution of one or more of the amino acids in the natural L-isomeric form with amino acids in the D-isomeric form. Thus, the peptide may include one or more D-amino acid resides, or may comprise amino acids which are all in the D-form. Retro-inverso forms of peptides in accordance with the present invention are also contemplated, for example, inverted peptides in which all amino acids are substituted with D-amino acid forms.

The skilled artisan will be aware that, in general, amino acid substitutions in a peptide typically involve the replacement of an amino acid with another amino acid of relatively similar properties (i.e., conservative amino acid substitutions). The properties of the various amino acids and effect of amino acid substitution on protein structure and function have been the subject of extensive study and knowledge in the art. For example, one can make the following isosteric and/or conservative amino acid changes in the parent polypeptide sequence with the expectation that the resulting polypeptides would have a similar or improved profile of the properties described above:

Substitution of alkyl-substituted hydrophobic amino acids: including alanine, leucine, isoleucine, valine, norleucine, S-2-aminobutyric acid, S-cyclohexylalanine or other simple alpha-amino acids substituted by an aliphatic side chain from C1-10 carbons including branched, cyclic and straight chain alkyl, alkenyl or alkynyl substitutions.

Substitution of aromatic-substituted hydrophobic amino acids: including phenylalanine, tryptophan, tyrosine, biphenylalanine, 1-naphthylalanine, 2-naphthylalanine, 2-benzothienylalanine, 3-benzothienylalanine, histidine, amino, alkylamino, dialkylamino, aza, halogenated (fluoro, chloro, bromo, or iodo) or alkoxy-substituted forms of the previous listed aromatic amino acids, illustrative examples of which are: 2-,3- or 4-aminophenylalanine, 2-,3- or 4-chlorophenylalanine, 2-,3- or 4-methylphenylalanine, 2-,3- or 4-methoxyphenylalanine, 5-amino-, 5-chloro-, 5-methyl- or 5-methoxytryptophan, 2′-, 3′-, or 4′-amino-, 2′-, 3′-, or 4′-chloro-, 2,3, or 4-biphenylalanine, 2′,-3′,- or 4′-methyl-2, 3 or 4-biphenylalanine, and 2- or 3-pyridylalanine.

Substitution of amino acids containing basic functions: including arginine, lysine, histidine, ornithine, 2,3-diaminopropionic acid, homoarginine, alkyl, alkenyl, or aryl-substituted (from C₁-C₁₀ branched, linear, or cyclic) derivatives of the previous amino acids, whether the substituent is on the heteroatoms (such as the alpha nitrogen, or the distal nitrogen or nitrogens, or on the alpha carbon, in the pro-R position for example. Compounds that serve as illustrative examples include: N-epsilon-isopropyl-lysine, 3-(4-tetrahydropyridyl)-glycine, 3-(4-tetrahydropyridyl)-alanine, N,N-gamma, gamma′-diethyl-homoarginine. Included also are compounds such as alpha methyl arginine, alpha methyl 2,3-diaminopropionic acid, alpha methyl histidine, alpha methyl ornithine where alkyl group occupies the pro-R position of the alpha carbon. Also included are the amides formed from alkyl, aromatic, heteroaromatic (where the heteroaromatic group has one or more nitrogens, oxygens, or sulfur atoms singly or in combination) carboxylic acids or any of the many well-known activated derivatives such as acid chlorides, active esters, active azolides and related derivatives) and lysine, ornithine, or 2,3-diaminopropionic acid.

Substitution of acidic amino acids: including aspartic acid, glutamic acid, homoglutamic acid, tyrosine, alkyl, aryl, arylalkyl, and heteroaryl sulfonamides of 2,4-diaminopriopionic acid, ornithine or lysine and tetrazole-substituted alkyl amino acids.

Substitution of side chain amide residues: including asparagine, glutamine, and alkyl or aromatic substituted derivatives of asparagine or glutamine.

Substitution of hydroxyl containing amino acids: including serine, threonine, homoserine, 2,3-diaminopropionic acid, and alkyl or aromatic substituted derivatives of serine or threonine. It is also understood that the amino acids within each of the categories listed above can be substituted for another of the same group.

For example, the hydropathic index of amino acids may be considered (Kyte & Doolittle, 1982, J. Mol. Biol., 157:105-132). The relative hydropathic character of the amino acid contributes to the secondary structure of the resultant protein, which in turn defines the interaction of the protein with other molecules. Each amino acid has been assigned a hydropathic index on the basis of its hydrophobicity and charge characteristics (Kyte & Doolittle, 1982), these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5). In making conservative substitutions, the use of amino acids whose hydropathic indices are within +/−2 is preferred, within +/−1 are more preferred, and within +/−0.5 are even more preferred.

Amino acid substitution may also take into account the hydrophilicity of the amino acid residue (e.g., U.S. Pat. No. 4,554,101). Hydrophilicity values have been assigned to amino acid residues: arginine (+3.0); lysine (+3.0); aspartate (+3.0); glutamate (+3.0); serine (+0.3); asparagine (+0.2); glutamine (+0.2); glycine (0); threonine (−0.4); proline (−0.5.+−0.1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). Replacement of amino acids with others of similar hydrophilicity is preferred.

Other considerations include the size of the amino acid side chain. For example, it would generally not be preferred to replace an amino acid with a compact side chain, such as glycine or serine, with an amino acid with a bulky side chain, e.g., tryptophan or tyrosine. The effect of various amino acid residues on protein secondary structure is also a consideration. Through empirical study, the effect of different amino acid residues on the tendency of protein domains to adopt an alpha-helical, beta-sheet or reverse turn secondary structure has been determined and is known in the art (see, e.g., Chou & Fasman, 1974, Biochemistry, 13:222-245; 1978, Ann. Rev. Biochem., 47: 251-276; 1979, Biophys. J., 26:367-384).

Based on such considerations and extensive empirical study, tables of conservative amino acid substitutions have been constructed and are known in the art. For example: arginine and lysine; glutamate and aspartate; serine and threonine; glutamine and asparagine; and valine, leucine and isoleucine. Alternatively: Ala (A) leu, ile, val; Arg (R) gln, asn, lys; Asn (N) his, asp, lys, arg, gln; Asp (D) asn, glu; Cys (C) ala, ser; Gln (Q) glu, asn; Glu (E) gln, asp; Gly (G) ala; His (H) asn, gln, lys, arg; Ile (I) val, met, ala, phe, leu; Leu (L) val, met, ala, phe, ile; Lys (K) gln, asn, arg; Met (M) phe, ile, leu; Phe (F) leu, val, ile, ala, tyr; Pro (P) ala; Ser (S), thr; Thr (T) ser; Trp (W) phe, tyr; Tyr (Y) trp, phe, thr, ser; Val (V) ile, leu, met, phe, ala.

Other considerations for amino acid substitutions include whether or not the residue is located in the interior of a protein or is solvent exposed. For interior residues, conservative substitutions would include: Asp and Asn; Ser and Thr; Ser and Ala; Thr and Ala; Ala and Gly; Ile and Val; Val and Leu; Leu and Ile; Leu and Met; Phe and Tyr; Tyr and Trp. (See, e.g., PROWL Rockefeller University website). For solvent exposed residues, conservative substitutions would include: Asp and Asn; Asp and Glu; Glu and Gln; Glu and Ala; Gly and Asn; Ala and Pro; Ala and Gly; Ala and Ser; Ala and Lys; Ser and Thr; Lys and Arg; Val and Leu; Leu and Ile; Ile and Val; Phe and Tyr. (Id.) Various matrices have been constructed to assist in selection of amino acid substitutions, such as the PAM250 scoring matrix, Dayhoff matrix, Grantham matrix, McLachlan matrix, Doolittle matrix, Henikoff matrix, Miyata matrix, Fitch matrix, Jones matrix, Rao matrix, Levin matrix and Risler matrix (Idem.)

In determining amino acid substitutions, one may also consider the existence of intermolecular or intramolecular bonds, such as formation of ionic bonds (salt bridges) between positively charged residues (e.g., His, Arg, Lys) and negatively charged residues (e.g., Asp, Glu) or disulfide bonds between nearby cysteine residues.

Methods of substituting any amino acid for any other amino acid in an encoded peptide sequence are well known and a matter of routine experimentation for the skilled artisan, for example by the technique of site-directed mutagenesis or by synthesis and assembly of oligonucleotides encoding an amino acid substitution and splicing into an expression vector construct.

Linkers

Additionally, modifications encompassed by the invention include introduction of linkers or spacers between the targeting sequence of the binding moiety or binding polypeptide and the detectable label or therapeutic agent. For example, use of such linkers/spacers can improve the relevant properties of the binding peptides (e.g., increase serum stability, etc.). These linkers can include, but are not restricted to, substituted or unsubstituted alkyl chains, polyethylene glycol derivatives, amino acid spacers, sugars, or aliphatic or aromatic spacers common in the art.

For example, suitable linkers include homobifunctional and heterobifunctional cross-linking molecules. The homobifunctional molecules have at least two reactive functional groups, which are the same. The reactive functional groups on a homobifunctional molecule include, for example, aldehyde groups and active ester groups. Homobifunctional molecules having aldehyde groups include, for example, glutaraldehyde and subaraldehyde.

Homobifunctional linker molecules having at least two active ester units include esters of dicarboxylic acids and N-hydroxysuccinimide. Some examples of such N-succinimidyl esters include disuccinimidyl suberate and dithio-bis-(succinimidyl propionate), and their soluble bis-sulfonic acid and bis-sulfonate salts such as their sodium and potassium salts.

Heterobifunctional linker molecules have at least two different reactive groups. Some examples of heterobifunctional reagents containing reactive disulfide bonds include N-succinimidyl 3-(2-pyridyl-dithio)propionate (Carlsson et al., 1978. Biochem. J., 173:723-737), sodium S-4-succinimidyloxycarbonyl-alpha-methylbenzylthiosulfate, and 4-succinimidyloxycarbonyl-alpha-methyl-(2-pyridyldithio)toluene. N-succinimidyl 3-(2-pyridyldithio)propionate is preferred. Some examples of heterobifunctional reagents comprising reactive groups having a double bond that reacts with a thiol group include succinimidyl 4-(N-maleimidomethyl)cyclohexahe-1-carboxylate and succinimidyl m-maleimidobenzoate. Other heterobifunctional molecules include succinimidyl 3-(maleimido)propionate, sulfosuccinimidyl 4-(p-maleimido-phenyl)butyrate, sulfosuccinimidyl 4-(N-maleimidomethyl-cyclohexane)-1-carboxylate, maleimidobenzoyl-5N-hydroxy-succinimide ester.

Furthermore, linkers that are combinations of the molecules and/or moieties described above, can also be employed to confer special advantage to the properties of the peptide. Lipid molecules with linkers may be attached to allow formulation of ultrasound bubbles, liposomes or other aggregation based constructs. Such constructs could be employed as agents for targeting and delivery of a diagnostic reporter, a therapeutic agent (e.g., a chemical “warhead” for therapy), or a combination of these.

Constructs employing dimers, multimers, or polymers of one or more peptide ligands of the invention are also contemplated. Indeed, there is ample literature evidence that the binding of low potency peptides or small molecules can be substantially increased by the formation of dimers and multimers. Thus, dimeric and multimeric constructs (both homogeneous and heterogeneous) are within the scope of the instant invention. The polypeptide sequences in the dimeric constructs can be attached at their N- or C-terminus or the N-epsilon nitrogen of a suitably placed lysine moiety (or another function bearing a selectively derivatizable group such as a pendant oxyamino or other nucleophilic group), or can be joined together via one or more linkers (e.g., those discussed herein) employing the appropriate attachment chemistry. This coupling chemistry can include amide, urea, thiourea, oxime, or aminoacetylamide (from chloro- or bromoacetamide derivatives, but is not so limited). Linkers can also be used for attachment to a chelating agent.

Therapeutic Agents

In other embodiments, therapeutic agents, including, but not limited to, cytotoxic agents, anti-angiogenic agents, pro-apoptotic agents, antibiotics, hormones, hormone antagonists, chemokines, drugs, prodrugs, toxins, enzymes or other agents may be used as adjunct therapies when using the antibody/peptide ligand complexes described herein. Drugs useful in the invention may, for example, possess a pharmaceutical property selected from the group consisting of antimitotic, antikinase, alkylating, antimetabolite, antibiotic, alkaloid, anti-angiogenic, pro-apoptotic agents and combinations thereof.

Imaging and Diagnostic Agents

Diagnostic agents are selected from, for example, the group consisting of a radionuclide, a radiological contrast agent, a paramagnetic ion, a metal, a fluorescent label, a chemiluminescent label, an ultrasound contrast agent and a photoactive agent. Such diagnostic agents are well known and any such known diagnostic agent may be used. Non-limiting examples of diagnostic agents may include a radionuclide such as ¹¹⁰In, ¹¹¹In, ¹⁷⁷Lu, ¹⁸F, ⁵²Fe, ⁶²Cu, ⁶⁴Cu, ⁶⁷Cu, ⁶⁷Ga, ⁶⁸Ga, ⁸⁶Y, ⁹⁰Y, ⁸⁹Zr, ^{94m}Tc, ⁹⁴Tc, ^{99m}Tc, ¹²⁰I, ¹²³I, ¹²⁴I, ¹²⁵I, ¹³¹I, ¹⁵⁴⁻¹⁵⁸Gd, ³²P, ¹¹C, ¹³N, ¹⁵O, ¹⁸⁶Re, ¹⁸⁸Re, ⁵¹Mn, ⁵²mMn, ⁵⁵Co, ⁷²As, ⁷⁵Br, ⁷⁶Br, ⁸²mRb, ⁸³Sr, or other gamma-, beta-, or positron-emitters. Paramagnetic ions of use may include chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) or erbium (III). Metal contrast agents may include lanthanum (III), gold (III), lead (II) or bismuth (III). Ultrasound contrast agents may comprise liposomes, such as gas filled liposomes. Radiopaque diagnostic agents may be selected from compounds, barium compounds, gallium compounds, and thallium compounds. A wide variety of fluorescent labels are known in the art, including but not limited to fluorescein isothiocyanate, rhodamine, phycoerythrin, phycocyanin, allophycocyanin, o-phthaldehyde and fluorescamine. Chemiluminescent labels of use may include luminol, isoluminol, an aromatic acridinium ester, an imidazole, an acridinium salt or an oxalate ester.

The inclusion of an isotopic form of one or more atoms in a molecule that is different from the naturally occurring isotopic distribution of the atom in nature is referred to as an “isotopically labeled form” of the molecule. All isotopic forms of atoms are included as options in the composition of any molecule, unless a specific isotopic form of an atom is indicated. For example, any hydrogen atom or set thereof in a molecule can be any of the isotopic forms of hydrogen, i.e., protium (¹H), deuterium (²H), or tritium (³H) in any combination. Similarly, any carbon atom or set thereof in a molecule can be any of the isotopic form of carbons, such as ¹¹C, ¹²C, ¹³C, or ¹⁴C, or any nitrogen atom or set thereof in a molecule can be any of the isotopic forms of nitrogen, such as ¹³N, ¹⁴N, or ¹⁵N. A molecule can include any combination of isotopic forms in the component atoms making up the molecule, the isotopic form of every atom forming the molecule being independently selected. In a multi-molecular sample of a compound, not every individual molecule necessarily has the same isotopic composition. For example, a sample of a compound can include molecules containing various different isotopic compositions, such as in a tritium or ¹⁴C radiolabeled sample where only some fraction of the set of molecules making up the macroscopic sample contains a radioactive atom. It is also understood that many elements that are not artificially isotopically enriched themselves are mixtures of naturally occurring isotopic forms, such as ¹⁴N and ¹⁵N, ³²S and ³⁴S, and so forth. A molecule as recited herein is defined as including isotopic forms of all its constituent elements at each position in the molecule. As is well known in the art, isotopically labeled compounds can be prepared by the usual methods of chemical synthesis, except substituting an isotopically labeled precursor molecule. The isotopes, radiolabeled or stable, can be obtained by any method known in the art, such as generation by neutron absorption of a precursor nuclide in a nuclear reactor, by cyclotron reactions, or by isotopic separation such as by mass spectrometry. The isotopic forms are incorporated into precursors as required for use in any particular synthetic route. For example, ¹⁴C and ³H can be prepared using neutrons generated in a nuclear reactor. Following nuclear transformation, ¹⁴C and ³H are incorporated into precursor molecules, followed by further elaboration as needed.

Peptides may advantageously be chemically synthesized and may optionally be (partially) overlapping and/or may also be ligated to other molecules, peptides, or proteins. Peptides may also be fused to form synthetic proteins, as in Welters et al. (Vaccine. 2004 Dec. 2; 23(3):305-11). It may also be advantageous to add to the amino- or carboxy-terminus of the peptide chemical moieties or additional (modified or D-) amino acids in order to increase the stability and/or decrease the biodegradability of the peptide. To enhance the solubility of the peptide, addition of charged or polar amino acids may be used, in order to enhance solubility and increase stability in vivo.

Amino acid mimetics may also be incorporated in the polypeptides. An “amino acid mimetic” as used here is a moiety other than a naturally occurring amino acid that conformationally and functionally serves as a substitute for an amino acid in a polypeptide of the present invention. Such a moiety serves as a substitute for an amino acid residue if it does not interfere with the ability of the peptide to elicit its desired activity. Amino acid mimetics may include non-protein amino acids. A number of suitable amino acid mimetics are known to the skilled artisan, they include cyclohexylalanine, 3-cyclohexylpropionic acid, L-adamantyl alanine, adamantylacetic acid and the like. Peptide mimetics suitable for peptides of the present invention are discussed by Morgan and Gainor, (1989) Ann. Repts. Med. Chem. 24:243-252.

The invention is now described with reference to the following Examples. Without further description, it is believed that one of ordinary skill in the art can, using the preceding description and the following illustrative examples, make and utilize the present invention and practice the claimed methods. The following working examples therefore, are provided for the purpose of illustration only and specifically point out the preferred embodiments of the present invention, and are not to be construed as limiting in any way the remainder of the disclosure. Therefore, the examples should be construed to encompass any and all variations which become evident as a result of the teaching provided herein.

### EXAMPLES

Case of Pituitary Apoplexy

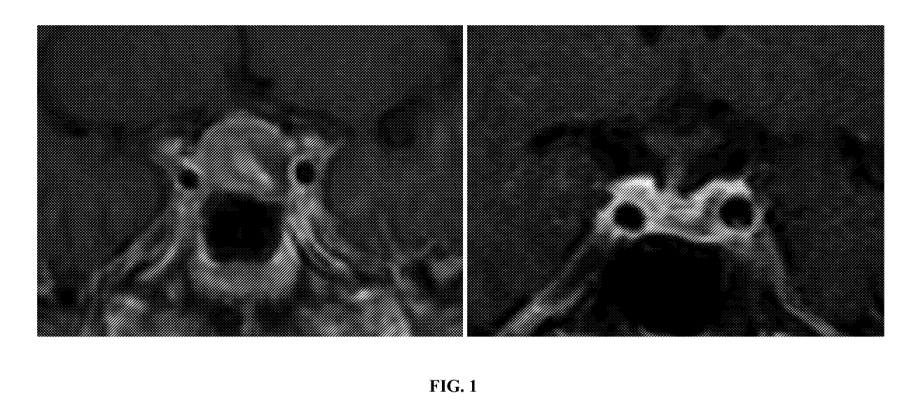

A 57 year-old male had the sudden onset of headache followed by excessive thirst, polydipsia, loss of energy, and generalized weakness. Pituitary MRI performed 4 days after the onset of his symptoms demonstrated findings consistent with apoplexy of a pituitary macroadenoma. Endocrine testing demonstrated panhypopituitarism. He was treated with glucocorticoids and without surgery. On MRI five months later there was no evidence of residual tumor. FIG. 1 (four panels) demonstrates an image from MRIs obtained 4 days after the onset of symptoms of pituitary apoplexy in a 57 year-old male (left, anteroposterior view). Repeat MRI five months later (right image of FIG. 1) shows no evidence of residual tumor.

Arguments Supporting the Proposed Mechanism

Pituitary Tumors have High-Energy Demand/Consumption

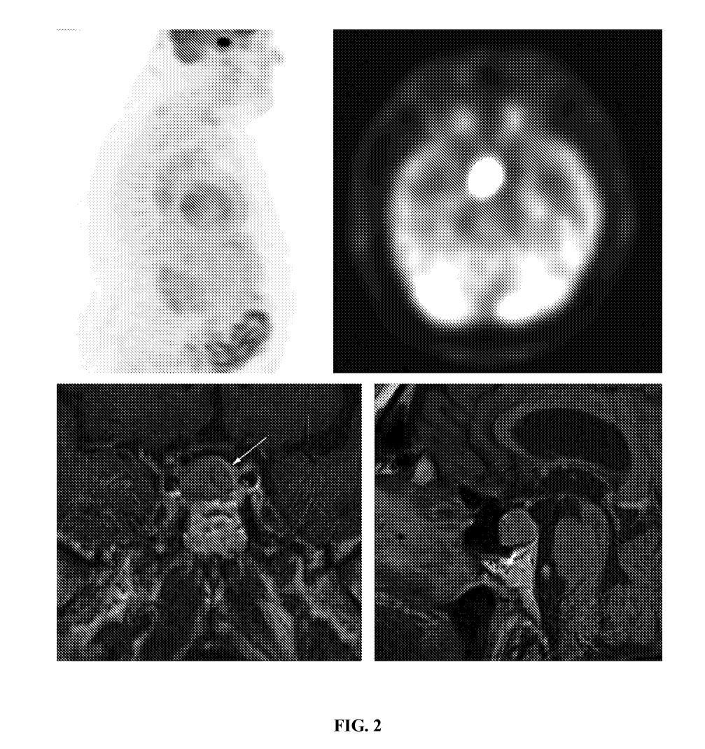

The total body [¹⁸F]-fluorodeoxyglucose(FDG)-PET and pituitary MRI shown in FIG. 2 (four panels) are of a 62 year old man in whom mediastinal lymphadenopathy was being investigated. The images demonstrate a pituitary macroadenoma that was not causing symptoms. Assessment indicated that this was a nonfunctioning pituitary adenoma.

The introduction of clinical positron emission tomography (PET) and the frequent use of FDG to study tumors and their metabolism demonstrated that pituitary adenomas consume a large amount of glucose in comparison to the surrounding brain, uptake exceeding that which occurs with other benign CNS tumors.^{4,5,13,16,33} Further, using PET Muhr et al., and others, demonstrated consistent high uptake of [¹¹C]-L-methionine by pituitary adenomas of various types.^{6,26} This high uptake of these metabolic tracers occurs with micro- and macroadenomas as well as secretory and nonfunctioning tumors.¹⁶ In contrast, the normal pituitary is not visualized as a region of increased uptake on FDG-PET.¹⁶ This elevated demand for glucose and methionine by pituitary adenomas is reduced by therapies that alter the tumor's hormone secretion (medical therapy with agonists of dopamine or somatostatin)^{4,6,16,26} or viability (radiation therapy).¹⁶

Pituitary Tumors have Limited Expression of Angiogenic Factors, Reduced Density of Vascularity, Limited Blood Supply, and Increased Intratumoral Pressure

Pituitary tumors have limited blood supply compared to other types of primary CNS tumors. This was recognized in the era in which arteriography was commonly used to assess patients with pituitary tumors. Pituitary adenomas show no “blush” on cerebral arteriography; in most instances, they have no visible blood supply detectable with arteriography. It had been noted even before the introduction of arteriography that at surgery pituitary macroadenomas tended to be relatively avascular compared to other types of CNS tumors. With the introduction of contrast-enhanced MRI, it became apparent that these tumors almost universally have reduced contrast enhancement compared to the normal gland, which is consistently seen as a brightly enhancing tissue relative to the limited enhancement of the adjacent pituitary tumor (FIG. 2, four panels). Further, the introduction of dynamic-enhanced imaging permits visualization of the blood flow to the pituitary gland and the tumor; it clearly and consistently demonstrates earlier and greater blood flow to the pituitary gland than to the tumor.

In our study of vascular endothelial growth factor (VEGF) expression by various types of CNS tumors using an RNase protection assay, we examined 10 pituitary adenomas (four Cushing's disease and one Nelson's corticotroph adenoma, three somatotroph adenomas, one thyrotropin-secreting adenoma, and one nonsecreting adenoma). We found increased expression of VEGF mRNA compared to normal brain in only 2 of the 10 pituitary tumors.⁷ In fact, in the pituitary adenomas the mean level of VEGF mRNA expression detected by RPCR was similar to the negligible level expressed by normal brain.

Consistent with this is reduced angiogenesis in pituitary tumors.^{18,34,40} This reduced density of microvasculature appears to have been first noted by Schechter³⁴ and was later confirmed in a series of studies using special stains by Turner et al., who examined the microvessel density of pituitary tumors by counting vessels labeled with endothelial markers (using antibodies to CD31, factor eight-related antigen, and biotinylated Ulex europaeus).⁴² Schechter, Jugenburg et al., and Turner et al. all observed less dense vessel representation in pituitary tumors than in the normal pituitary,^{18,34,42} which is distinctly different from other tumor types that have been studied similarly.^{11,18,40,41,43} For instance, in contrast to the circumstance with pituitary tumors, benign and precancerous lesions of the breast have a greater density of microvasculature than the normal host tissue.¹¹

Thus, available evidence indicates that there is limited angiogenesis and reduced vessel density in pituitary adenomas, features upon which excess pressure might act to further diminish perfusion of the adenoma.

Available evidence indicates high intratumoral pressure in large and small adenomas. At surgery, high intrasellar pressure (20-40 mm Hg) has been measured consistently in patients with pituitary macroadenomas.^{1,19-21} Measurement of blood flow in pituitary tumors versus normal gland using the xenon washout technique during surgery demonstrates very low blood flow in the tumor compared to the normal gland.¹⁹ Further, the development of a histological pseudocapsule, a result of the effect of increased pressure within an adenoma on the surrounding normal pituitary gland, begins to occur with tumors as small as 1-2 mm and occurs in essentially all micro- and macroadenomas.²⁷ This increased pressure within an adenoma, combined with intrinsically limited vascularity, reduces the perfusion of the tumor and enhances the susceptibility of it to ischemia and to infarction and occurs in large and small tumors (see below).^{24,28,37}

Circumstances Precipitating Apoplexy of Pituitary Adenomas

Several clinical situations have long been known to be associated with induction of apoplexy of pituitary adenomas. These include events that acutely diminish blood pressure or plasma glucose and events that increase tumor metabolism and demand for blood flow.

The most common circumstances that acutely precipitate pituitary apoplexy are events that alter systemic blood pressure, such as cardiac surgery and myocardial infarction.^{2,10,15,25,35,38} Traumatic injury associated with shock, aortic dissection, and other types of surgery have also been linked with the onset of pituitary apoplexy.^{10,35,37,39,45}

Events that alter the balance between glucose supply and metabolic demand, such as hypoglycemia associated with insulin tolerance testing, or increasing metabolic demand by stimulation testing with hypothalamic releasing factors, have been reported to precipitate apoplexy of pituitary tumors.^{8,10,31,44,46}

### Experiments to Assess Vulnerability of Pituitary Tumor Cells to Hypoglycemia

To examine the tolerance of fresh pituitary tumor cells to glucose deprivation compared to normal cells we performed the following experiments.

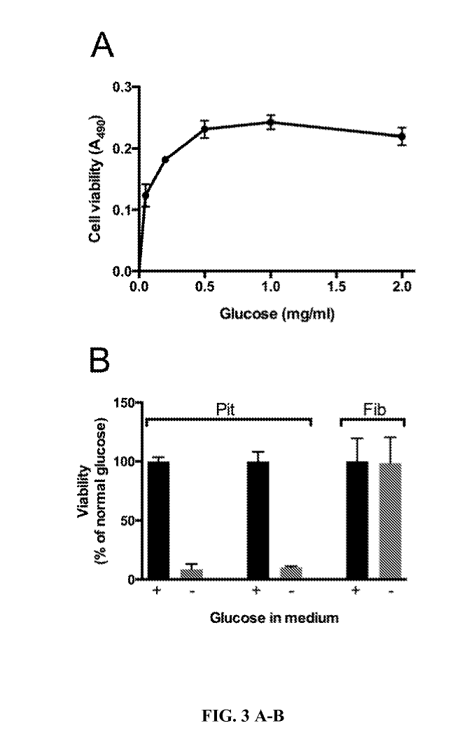

Pituitary tumors for laboratory investigation were obtained under an NINDS IRB-approved protocol, 03-N-0164: Evaluation and Treatment of Neurosurgical Disorders. Pituitary tumor cell cultures (from one ACTH-secreting, one growth hormone secreting, and one non-secreting tumor) were prepared by enzymatic digestion as previously described³⁰ with minor modifications. Red blood cells were removed by subjecting the cell suspension to Lymphocyte Separation Medium. Tumor cells were cultured in DMEM-10% FCS for 24 hours. Tumor cells were then plated in serum-free medium with or without glucose for 20 hours. In 3/3 freshly prepared cultures, pituitary tumor cells were unable to survive in the absence of glucose (FIG. 3A-B). By contrast, cultured human fibroblasts (FIG. 3B) were still viable after 20 hours in the absence of glucose.

These results are consistent with the hypothesis that pituitary adenoma cells are particularly sensitive to glucose deprivation. Whether this sensitivity results from unusually high-energy demand or from an inability to utilize alternative energy sources, or some combination of these two possibilities, remains to be determined.

### Discussion

Patients with the typical clinical presentation of apoplexy of a pituitary adenoma generally have macroadenomas and complain of headache, generalized weakness, loss of vision caused by compression of their optic nerves or chiasm, and diplopia caused by dysfunction of the cranial nerves controlling ocular movement. Further, ischemic infarction can occur and produce symptoms and signs in microadenomas.²⁸ It also occurs silently without production of symptoms, as shown by histological features consistent with apoplexy in as many as 25% of microadenomas removed surgically.^{24,37}

Previously proposed mechanisms for pituitary apoplexy include reduced blood supply to the tumor produced by events such as hypotension, rapid growth outpacing the development of adequate blood supply to the tumor,¹² direct pressure by the tumor on the portal vessels or the hypophyseal arteries causing acute ischemia of the tumor,³² increased intratumoral pressure which itself acutely impairs the blood flow to the tumor,⁴⁷ increased metabolic activity beyond adequate arterial supply after stimulation with hypothalamic releasing factors,³¹ and hemorrhage resulting from fragility of the tumor vessels.¹⁰

There is evidence supporting these proposed mechanisms as contributing factors in acute pituitary apoplexy. Increased intrasellar pressure has been documented consistently by measurements made at surgery in patients with macroadenomas.^{1,19,21} It has also been established in patients with pituitary apoplexy,⁴⁷ although it cannot be known if the increased pressure was a consequence of the apoplexy or the cause of it. Thus, the consistency of the pressure measurements and the nearly universal production of a histological pseudocapsule by the intratumoral pressure in small and large tumors,²⁷ increased pressure within the tumor that alters perfusion of it and associated limited vascularity (see above) are baseline circumstances in all of these tumors. Something else seems to be needed to set the stage for infarction, with the acute clinical event or with clinically silent infarction.

Although the consistent high uptake of glucose and methionine by pituitary adenomas and the reduced angiogenesis, reduced microvascular density and blood flow in pituitary tumors has been known for some time, it is surprising that none of these features have previously been proposed as setting the stage for spontaneous tumor infarction, which occurs more frequently in pituitary tumors than any other tumor of the central nervous system. We offer that apoplexy of a pituitary adenoma is the product of intrinsic features of these tumors leaving the tumor in a state of tenuous balance between high metabolic demand, which, based on PET imaging, exists with large and small adenomas, and marginal blood supply, which exists in large and small tumors, in relation to that demand. This would make the tumor vulnerable to acute ischemia by any general event that acutely alters the balance between blood flow and metabolism, such as systemic hypotension, decreased supply of nutrients, such as hypoglycemia with insulin administration, or increasing the tumor's metabolic demand with administration of hypothalamic releasing factors. Our laboratory examination of the vulnerability of these tumors to diminished glucose indicates their peculiar susceptibility to deprivation of nutrients such as glucose. Although an increase in this imbalance can be precipitated by abrupt events that alter blood supply to the tumor, such as hypotension associated with surgery, etc., or increased metabolic demand by the tumor after administration of hypothalamic releasing factors, by virtue of these intrinsic features of pituitary tumors a baseline imbalance exists even without these precipitating events. In fact, most pituitary apoplexy occurs in the absence of one of the known acute predisposing events, perhaps as a result of spontaneous fluctuations in the metabolic activity or intratumoral pressure and perfusion in individual tumors that are teetering on a precarious balance of high metabolic demand and limited perfusion. Finally, the mechanism proposed also might explain why it is that the tumor is selectively vulnerable to infarction compared to the normal gland.

The mechanism that we propose here offers therapeutic opportunities to induce selective infarction of an adenoma. These include manipulation of perfusion of the tumor with controlled systemic hypotension, controlled hypoglycemia using graded doses of insulin, or by using intravenous deoxyglucose to compete with blood glucose, or intravenous administration of selected hypothalamic releasing factors, used alone or in various combinations, to tip the balance in favor of ischemia selective to the tumor. Any of these approaches would need to be studied initially with smaller tumors which were refractory to standard therapies, to avoid the known risks of inducing pituitary apoplexy in a larger tumor. The clinical possibilities of this strategy will require appropriate preclinical investigations to investigate safety issues as well antitumor potential. In this respect, it is noteworthy that many patients with pituitary apoplexy can be managed successfully with conservative, non-surgical therapy with the same general success with long-term tumor control as occurs in surgical patients.^{3,17,37} It should also be noted that this approach, if successful, would provide the potential of a tumoricidal treatment of these tumors, rather than the tumor stabilizing effect of current medical therapies.

The disclosures of each and every patent, patent application, and publication cited herein are hereby incorporated by reference herein in their entirety.

Headings are included herein for reference and to aid in locating certain sections. These headings are not intended to limit the scope of the concepts described therein under, and these concepts may have applicability in other sections throughout the entire specification.

While this invention has been disclosed with reference to specific embodiments, it is apparent that other embodiments and variations of this invention may be devised by others skilled in the art without departing from the true spirit and scope of the invention.

### BIBLIOGRAPHY

1. Arafah B M, Prunty D, Ybarra J, Hlavin M L, Selman W R: The dominant role of increased intrasellar pressure in the pathogenesis of hypopituitarism, hyperprolactinemia, and headaches in patients with pituitary adenomas. J Clin Endocrinol Metab 85:1789-1793, 2000
2. Armstrong M R, Douek M, Schellinger D, Patronas N: Regression of pituitary macroadenoma after pituitary apoplexy: CT and MR studies. J Comput Assist Tomogr 15:832-834, 1991
3. Ayuk J, McGregor E J, Mitchell R D, Gittoes N J: Acute management of pituitary apoplexy—surgery or conservative management? Clin Endocrinol (Oxf) 61:747-752, 2004
4. Bergström M, Muhr C, Lundberg P O, Bergström K, Gee A D, Fasth K J, et al: Rapid decrease in amino acid metabolism in prolactin-secreting pituitary adenomas after bromocriptine treatment: a PET study. J Comput Assist Tomogr 11:815-819, 1987
5. Bergström M, Muhr C, Lundberg P O, Bergström K, Lundqvist H, Antoni G, et al: Amino acid distribution and metabolism in pituitary adenomas using positron emission tomography with D-[11C]methionine and L-[11C]methionine. J Comput Assist Tomogr 11:384-389, 1987
6. Bergström M, Muhr C, Lundberg P O, Långström B: PET as a tool in the clinical evaluation of pituitary adenomas. J Nucl Med 32:610-615, 1991
7. Berkman R, Merrill M, Reinhold W, Monacci W, Saxena A, Clark W, et al: Expression of the vascular permeability factor/vascular endothelial growth factor gene in central nervous system neoplasms. J Clin Invest 91:153, 1993
8. Bernstein M, Hegele R A, Gentili F, Brothers M, Holgate R, Sturtridge W C, et al: Pituitary apoplexy associated with a triple bolus test. Case report. J Neurosurg 61:586-590, 1984
9. Bills D C, Meyer F B, Laws E R J, Davis D H, Ebersold M J, Scheithauer B W, et al: A Retrospective Analysis of Pituitary Apoplexy. Neurosurgery 33:602-609, 1993
10. Biousse V, Newman N J, Oyesiku N M: Precipitating factors in pituitary apoplexy. J Neurol Neurosurg and Psychiatry 71:542-545, 2001
11. Brem S S, Jensen H M, Gullino P M: Angiogenesis as a marker of preneoplastic lesions of the human breast. Cancer 41:239-244, 1978
12. Brougham M, Heusner A P, Adams R D: Acute degenerative changes in adenomas of the pituitary body-with special reference to pituitary apoplexy. J Neurosurg, 1950
13. De Souza B, Brunetti A, Fulham M J, Brooks R A, DeMichele D, Cook P, et al: Pituitary microadenomas: a PET study. Radiology 177:39-44, 1990
14. Dubuisson A S, Beckers A, Stevenaert A: Classical pituitary tumour apoplexy:

clinical features, management and outcomes in a series of 24 patients. Clin Neurol Neurosurg 109:63-70, 2007
15. Elsässer Imboden P N, de Tribolet N, Lobrinus A, Gaillard R C, Portmann L, Pralong F, et al: Apoplexy in pituitary macroadenoma: eight patients presenting in 12 months. Medicine 84:188-196, 2005
16. Francavilla T L, Miletich R S, DeMichele D, Patronas N J, Oldfield E H, Weintraub B D, et al: Positron emission tomography of pituitary macroadenomas: hormone production and effects of therapies. Neurosurgery 28:826-833, 1991
17. Gruber A, Clayton J, Kumar S, Robertson I, Howlett T A, Mansell P: Pituitary apoplexy: retrospective review of 30 patients-is surgical intervention always necessary? Br J Neurosurg 20:379-385, 2006
18. Jugenburg M, KOVACS K, Stefaneanu L, Scheithauer B W: Vasculature in Nontumorous Hypophyses, Pituitary Adenomas, and Carcinomas: A Quantitative Morphologic Study. Endocr Pathol 6:115-124, 1995
19. Kruse A, Astrup J, Cold G E, Hansen H H: Pressure and blood flow in pituitary adenomas measured during transsphenoidal surgery. Br J Neurosurg 6:333-341, 1992
20. Lees P D, Fahlbusch R, Zrinzo A, Pickard J D: Intrasellar pituitary tissue pressure, tumour size and endocrine status—an international comparison in 107 patients. Br J Neurosurg 8:313-318, 1994
21. Lees P D, Pickard J D: Hyperprolactinemia, intrasellar pituitary tissue pressure, and the pituitary stalk compression syndrome. J Neurosurg 67:192-196, 1987
22. Liu J K, Couldwell W T: Pituitary apoplexy in the magnetic resonance imaging era: clinical significance of sphenoid sinus mucosal thickening. J Neuosurg 104:892-898, 2006
23. Liu Z H, Chang C N, Pai P C, Wei K C, Jung S M, Chen N Y, et al: Clinical features and surgical outcome of clinical and subclinical pituitary apoplexy. J Clin Neurosci 17:694-699, 2010
24. Mohr G, Hardy J: Hemorrhage, necrosis, and apoplexy in pituitary adenomas. Surg Neurol 18:181-189, 1982
25. Moller-Goede D L, Brandle M, Landau K, Bernays R L, Schmid C: Pituitary apoplexy: re-evaluation of risk factors for bleeding into pituitary adenomas and impact on outcome. Eur J Endocrinol 164:37-43, 2010
26. Muhr C: Positron emission tomography in acromegaly and other pituitary adenoma patients. Neuroendocrinology 83:205-210, 2006
27. Oldfield E H, Vortmeyer A O: Development of a histological pseudocapsule and its use as a surgical capsule in the excision of pituitary tumors. J Neurosurg 104:7-19, 2006
28. Randall B R, Couldwell W T: Apoplexy in pituitary microadenomas. Acta Neurochir 152:1737-1740, 2010
29. Randeva H S, Schoebel J, Byrne J, Esiri M, Adams C B, Wass J A: Classical pituitary apoplexy: clinical features, management and outcome. Clin Endocrinol (Oxf) 51:181-188, 1999
30. Renner U, Brockmeier S, Strasburger C J, Lange M, Schopohl J, Muller O A, et al: Growth hormone (GH)-releasing peptide stimulation of GH release from human somatotroph adenoma cells: interaction with GH-releasing hormone, thyrotropin-releasing hormone, and octreotide. J Clin Endocrinol Metab 78:1090-1096, 1994
31. Rotman Pikielny P, Patronas N, Papanicolaou D A: Pituitary apoplexy induced by corticotrophin-releasing hormone in a patient with Cushing&apos;s disease. Clin Endocrinol (Oxf) 58:545-549, 2003
32. Rovit R L, Fein J M: Pituitary apoplexy: a review and reappraisal. J Neurosurg 37:280-288, 1972
33. Ryu S I, Tafti B A, Skirboll S L: Pituitary adenomas can appear as hypermetabolic lesions in (18) F-FDG PET imaging. J Neuroimaging 20:393-396, 2010
34. Schechter J: Ultrastructural changes in the capillary bed of human pituitary tumors. Am J Pathol 67:109-126, 1972
35. Semple P L, Jane J A, Laws E R: Clinical relevance of precipitating factors in pituitary apoplexy. Neurosurgery 61:956-961-discussion 961-952, 2007
36. Semple P L, Jane J A, Lopes M B S, Laws E R: Pituitary apoplexy: correlation between magnetic resonance imaging and histopathological results. J Neurosurg 108:909-915, 2008
37. Sibal L, Ball S G, Connolly V, James R A, Kane P, Kelly W F, et al: Pituitary apoplexy: a review of clinical presentation, management and outcome in 45 cases. Pituitary 7:157-163, 2004
38. Turgut M, Mahapatra A K, Powell M, Muthukumar N: Pituitary Apoplexy, 2014
39. Turgut M, Özsunar Y, Ba [symbol] ak S, Güney E, Kir E, Meteo\check{g}lu İ: Pituitary apoplexy: an overview of 186 cases published during the last century. Acta Neurochir 152:749-761, 2010
40. Turner H E: Pituitary Tumor Angiogenesis. Endocrinologist 11:465-469, 2001
41. Turner H E, Nagy Z, Gatter K C, Esiri M M, Harris A L, Wass J A: Angiogenesis in pituitary adenomas—relationship to endocrine function, treatment and outcome. J Endocrinol 165:475-481, 2000
42. Turner H E, Nagy Z, Gatter K C, Esiri M M, Harris A L, Wass J A: Angiogenesis in pituitary adenomas and the normal pituitary gland. J Clin Endocrinol Metab 85:1159-1162, 2000
43. Turner H E, Nagy Z, Gatter K C, Esiri M M, Wass J A, Harris A L: Proliferation, bcl-2 expression and angiogenesis in pituitary adenomas: relationship to tumour behaviour. Br J Cancer 82:1441-1445, 2000
44. Vassallo M, Rana Z, Allen S: Pituitary apoplexy after stimulation tests. Postgrad Med J 70:444-445, 1994
45. Verrees M, Arafah B M, Selman W R: Pituitary tumor apoplexy: characteristics, treatment, and outcomes. Neurosurg Focus 16:E6, 2004
46. Yamamoto T, Yano S, Kuroda J-i, Hasegawa Y, Hide T, Kuratsu J-i: Pituitary Apoplexy Associated with Endocrine Stimulation Test: Endocrine Stimulation Test, Treatment, and Outcome. Case Rep Endocrinol 2012:1-6, 2012
47. Zayour D H, Selman W R, Arafah B M: Extreme elevation of intrasellar pressure in patients with pituitary tumor apoplexy: relation to pituitary function. J Clin Endocrinol Metab 89:5649-5654, 2004

What is claimed is:

1. A method for treating a pituitary adenoma by inducing infarction of said pituitary adenoma, said method comprising administering to a subject in need thereof a pharmaceutical composition comprising a pharmaceutically acceptable carrier and an effective amount of an agent selected from the group consisting of an agent that inhibits glucose uptake or glucose in pituitary adenoma cells, an agent that induces or controls hypoglycemia, an agent that controls systemic hypotension, and a hypothalamic releasing factor, thereby treating said pituitary adenoma by inducing infarction of said pituitary adenoma.

2. The method of claim 1, wherein said agent that inhibits glucose uptake is a competitive inhibitor of glucose.

3. The method of claim 1, wherein said agent that inhibits glucose uptake is 2-deoxy-D-glucose (2DG).

4. The method of claim 3, wherein said 2DG is administered for at least two weeks.

5. The method of claim 3, wherein said 2DG is administered daily for at least three consecutive days.

6. The method of claim 3, wherein said 2DG is administered daily for at least ten consecutive days.

7. The method of claim 3, wherein said 2DG is administered daily for at least thirty consecutive days.

8. The method of claim 3, wherein said 2DG is administered at least once daily.

9. The method of claim 3, wherein said 2DG is administered at dose ranging from about 0.10 mg kg body weight to about 1 g/kg body weight.

10. The method of claim 3, further wherein an effective amount of metformin is administered.

11. The method of claim 1, wherein said agent that inhibits glucose uptake inhibits a glucose transporter.

12. The method of claim 11, wherein said glucose transporter (GLUT) is GLUT1 or GLUT3.

13. The method of claim 12, wherein said agent that inhibits said GLUT is phloretin, genistein, or silybin/silibinin.

14. The method of claim 1, wherein said agent that induces or controls hypoglycemia is insulin.

15. The method of claim 14, wherein said insulin is administered at a dose ranging from about 0.15 international units (IU)/kg body weight to about 20.0 IU/kg body weight.

16. The method of claim 15, wherein said insulin is administered at a dose selected from the group consisting of 0.15, 0.5, 1.0, 2.0, 5.0, 10.0, 15.0, and 20.0 IU/kg body weight.

17. The method of claim 1, wherein said pharmaceutical composition comprises a hypothalamic releasing factor selected from the group consisting of Thyrotropin-releasing hormone (TRH), Corticotropin-releasing hormone (CRH), Gonadotropin-releasing hormone (GnRH), and Growth hormone-releasing hormone (GHRH).

18. The method of claim 17, wherein said TRH is administered at a dose of about 200 micrograms (μg)/kg body weight.

19. The method of claim 17, wherein said CRH is administered at a dose of about 1.0 μg/kg body weight or a unit dose of about 100 μg.

20. The method of claim 17, wherein said GnRH is administered at a unit dose of about 100 μg.

21. The method of claim 17, wherein said GHRH is administered at a dose of about 1.0 μg/kg body weight.

22. The method of claim 17, wherein at least two different hypothalamic releasing factors are administered.

23. The method of claim 1, wherein said method that controls systemic hypotension comprises inducing deep anesthesia and heavy analgesia in said subject.

24. The method of claim 1, wherein said method that controls systemic hypotension comprises administration of standard anesthesia and administration of a hypotensive drug.

25. The method of claim 24, wherein said hypotensive drug is selected from the group consisting of sodium nitroprusside (SNP), nitroglycerin (NTG), trimethaphan, calcium channel antagonists, a β-adrenoceptor antagonist, an angiotensin converting enzyme (ACE) inhibitor, and an adrenoceptor agonist.

26. The method of claim 1, wherein said method that controls systemic hypotension reduces mean arterial blood pressure (MAP) by about 30-40% compared to the subject's normal MAP.

27. The method of claim 26, wherein said reduced MAP is at least about 50 mm Hg.

28. The method of claim 1, wherein at least two of said agents are administered.

29. The method of claim 1, wherein said pharmaceutical composition is administered systemically.

30. The method of claim 29, wherein said pharmaceutical composition is administered intravenously.

31. The method of claim 1, wherein said agent is administered in two or more cycles of treatment.

32. The method of claim 31, wherein said agent is administered at an increasing dose upon each of said cycles of treatment.
